(19) United States
(12) Patent Application Publication
(10) Pub. No.: US 2022/0402873 A1
(43) Pub. Date: Dec. 22, 2022

(54) NOVEL P62 LIGAND COMPOUND, AND COMPOSITION CONTAINING SAME FOR PREVENTING, AMELIORATING, OR TREATING PROTEINOPATHIES

(71) Applicants: AUTOTAC INC., Seoul (KR); SEOUL NATIONAL UNIVERSITY R&DB FOUNDATION, Seoul (KR)

(72) Inventors: Yong Tae KWON, Seoul (KR); Chang Hoon JI, Seoul (KR); Hyun Tae KIM, Seoul (KR); Jeong Eun NA, Gyeonggi-do (KR); Hee Yeon KIM, Gyeonggi-do (KR); Min Ju LEE, Gyeonggi-do (KR)

(73) Assignees: AUTOTAC INC., Seoul (KR); SEOUL NATIONAL UNIVERSITY R&DB FOUNDATION, Seoul (KR)

(21) Appl. No.: 17/761,830

(22) PCT Filed: Sep. 21, 2020
(86) PCT No.: PCT/KR2020/012715

§ 371 Date: Mar. 18, 2022

Related U.S. Application Data
(60) Provisional application No. 62/903,489, filed on Sep. 20, 2019.

(51) Int. Cl. C07D 213/64; C07D 213/69; C07D 239/34; C07D 213/74; C07D 207/335; C07D 333/32; C07D 307/58; C07D 307/52; C07D 213/65

(52) U.S. Cl. CPC C07D 213/64; C07D 213/69; C07D 239/34; C07D 213/74; C07D 207/335; C07D 333/32; C07D 307/58; C07D 307/52; C07D 213/65

(57) ABSTRACT

Novel p62 ligand compounds, or a stereoisomer, a solvate, a hydrate, or a prodrug thereof are disclosed. The novel compounds, stereoisomer, solvate, hydrate, or prodrug activates selective autophagy in cells to selectively remove proteins, organelles, and coagulations in the body, and thus can be advantageously used as a pharmaceutical composition for preventing, ameliorating, or treating various proteinopathies. Compositions such as pharmaceutical composition or food compositions containing the novel p62 ligand compounds, stereoisomer, solvate, hydrate, or prodrug thereof as well as uses thereof are disclosed.

### TECHNICAL FIELD

### Cross Reference Related Application

The present application claims the benefit of priority to U.S. Patent Application No. 62/903,489, filed Sep. 20, 2019, the entire contents of which is incorporated herein for all purposes by this reference.

The present invention relates to a novel p62 ligand compound, a pharmaceutical or food composition for preventing or treating proteinopathies comprising the same.

### BACKGROUND ART

The N-end rule pathway is a proteolytic system where a specific N-terminal residue of a protein acts as a decomposition signal (FIG. 1). As the decomposition signal of the N-end rule, there are type 1, which is base residues having N-terminal arginine (Nt-Arg), lysine (Nt-Lys) and histidine (Nt-His), and type 2, which is hydrophobic residues having phenylalanine (Nt-Phe), leucine (Nt-Leu), tryptophan (Nt-Trp), tyrosine (Nt-Tyr) and isoleucine (Nt-Ile). These N-terminal residues bind to ligands (referred to as N-ligand) of specific recognition elements (N-recognins).

The present inventors first discovered or cloned previously known N-lecognins, namely, UBR1, UBR2, UBR4, and UBR5, and found that they utilize the UBR box as a substrate recognition domain (Tasaki, T. et al., Mol Cell Biol 25, 7120-36 (2005))). The present inventors have also found that the UBR box binds to type-1 N-end rule ligands (Nt-Arg, Nt-Lys, Nt-His) such as the N-terminal Arg residue to recognize a substrate, and to link a ubiquitin chain to the substrate. It has further found that UBR1 and UBR2 have an N-domain which plays an important role in binding to type-2 N-end rule ligands (Nt-Trp, Nt-Phe, Nt-Tyr, Nt-Leu, Nt-Ile) (Sriram, S. M., Kim, B. Y. & Kwon, Y. T., Nat Rev Mol Cell Biol 12, 735-47 (2011)). The ubiquitinated substrate produced from the binding of N-lecognins to the N-end rule ligands is delivered to proteasome where it is degraded into a short peptide. In this process, specific N-terminal residues (Nt-Arg, Nt-His, Nt-Lys, Nt-Trp, Nt-Phe, Nt-Tyr, Nt-Leu, Nt-Leu) are the essential determinants of binding because N-recognins provide most of the hydrogen bonds required for targeting the N-end rule substrates (Sriram, S. M. & Kwon, Y. T., Nat Struct Mol Biol 17, 1164-5 (2010)).

Misfolded proteins that do not fold properly in cells are aggregated when left for a long time and become cytotoxic substances such as blocking proteasome or reducing other cellular functions, so they are ubiquitinated by ubiquitin ligase and delivered to the proteasome for degrading (Ji, C. H. & Kwon, Y. T., Mol Cells 40, 441-449 (2017)). In normal cells, this process is smooth and the damage caused by misfolded proteins is minimized, whereas in the elderly neurons this process is slow, ubiquitinated misfolded proteins are accumulated, and these excess protein wastes are converted back to aggregates (Ciechanover, A. & Kwon, Y. T., Exp Mol Med 47, e147 (2015)). In addition, among the proteinopathies, specific mutant proteins have a strong property of being transformed into aggregates in the neurons of patients suffering from degenerative brain diseases such as Huntington's disease, Parkinson's disease, human mad cow disease, Lou Gehrig's disease, and thus cannot be degraded into the above-described proteasome. Because the proteasome has a narrow inner diameter of 13 angstroms, the misfolded proteins must be unfolded, and when the proteins are aggregated, they cannot be unfolded.

Meanwhile, autophagy is a major intracellular protein degradation system along with the ubiquitin-proteasome system. Autophagy is a protein degradation process essential to maintain cell homeostasis and genetic stability by degrading aged or impaired cellular organelles or damaged or abnormally folded proteins (Ji, C. H. & Kwon, Y. T., Mol Cells 40, 441-449 (2017)). In particular, when the aggregates of misfolded proteins are accumulated in a cytoplasm, they become cytotoxic substances, so they should be received and degraded by autophagy. The mechanism of autophagy is largely divided into macroautophagy, microautophagy, and chaperone-mediated autophagy, and is divided into bulk autophagy and selective autophagy, depending on the purpose of degrading the intracellular substances. (Dikic, I. & Elazar, Z., Nat Rev Mol Cell Biol 19, 349-364 (2018)). Among them, selective autophagy and chaperone-mediated autophagy cause selective degradation of intracellular dysfunctional organelles or unwanted proteins. By inducing selective autophagy, the development of new therapies for diseases based on the accumulation of pathologically misfolded proteins and dysfunctional organelles is currently building a new paradigm. The p62/SQSTM1/Sequestosome-1 protein is important for initiating the formation of autophagosome, which is a mediator in the mechanism for selective autophagy, and delivering the contents. In this case, p62/SQRSM1/Sequestosome-1 binds to the misfolded protein and its aggregates and delivers them to the autophagosome. When delivering the misfolded proteins to the autophagosome, p62 undergoes self-oligomerization as a key process (Ji, C. H. & Kwon, Y. T., Mol Cells 40, 441-449 (2017)). At this time, the misfolded proteins are concentrated together to reduce the volume, thus facilitating degradation by autophagy. PB1 domain mediates the self-oligomerization of p62, but the regulatory mechanism thereof is not well known. The misfolded protein-p62 conjugate delivered to the autophagosome is degraded by lysosomal enzymes as the autophagosome binds to the lysosome. Through this mechanism, the autophagy is important for maintaining cell homeostasis through intracellular changes in damaged proteins and cellular organelles. When autophagic function is weakened, it leads to the accumulation and aggregation of the misfolded proteins, which results in proteinopathy. (Ciechanover, A. & Kwon, Y. T., Exp Mol Med 47, e147 (2015)). The core technology of the present invention is to provide a method for effectively removing misfolded protein or its aggregate that causes proteinopathy. To this end, it is necessary to activate only the selective autophagy without activating the bulk autophagy that has a wide range of effects on various biological pathways.

Research on activating autophagy to treat proteinopathy has been actively conducted. The regulator that normally inhibits the bulk autophagy is mTOR, and the method of activating the autophagy using mTOR inhibitors is the most widely used (Jung, C. H., Ro, S. H., Cao, J., Otto, N. M. & Kim, D. H., FEBS Lett 584, 1287-95 (2010)). Specifically, by using rapamycin, amyloid beta (Ab) and tau were eliminated and simultaneously cognitive ability was improved in an AD animal model overexpressing APP, (Caccamo, A., Majumder, S., Richardson, A., Strong, R. & Oddo, S., J Biol Chem 285, 13107-20 (2010)), tau was eliminated in an AD animal model overexpressing tau (Rodriguez-Navarro, J. A. et al., Neurobiol Dis 39, 423-38 (2010)), and the over-expressed mutant alpha-synuclein protein aggregate was eliminated in a PD mouse model (Webb, J. L., Ravikumar, B., Atkins, J., Skepper, J. N. & Rubinsztein, D. C., J Biol Chem 278, 25009-13 (2003)). It was confirmed that CCI-779, a rapamycin-like substance, is used to efficiently eliminate huntingtin aggregates and also to improve animal behavior and cognitive ability in a HD mouse (Ravikumar, B., Duden, R. & Rubinsztein, D. C., Hum Mol Genet 11, 1107-17 (2002)). However, mTOR plays a very important role in various intracellular pathways including NF-kB. Therefore, although it exhibits excellent activity to eliminate misfolded protein aggregates of proteinopathies, there is a limitation in that these bulk autophagy activators, which are known that mTOR is a drug target, are used as therapeutic agents.

As described above, currently, there is no therapeutic agents to treat most proteinopathies, and in the case of ubiquitin ligase ligand for elimination of the misfolded proteins that are the main cause, it is difficult to eliminate them when the misfolded proteins are aggregated. In addition, mTOR inhibitor, which is the most commonly used compound as a bulk autophagy activator, is inadequate as a therapeutic agent because mTOR inhibitors play a wide role in regulating overall gene expression in cells in response to stimuli from various external environments, in addition to the regulation of autophagy. Therefore, there is a need for developing a method for eliminating misfolded protein aggregates by activating p62, a key regulator of selective autophagy, without reducing the activity of mTOR, a regulator of bulk autophagy.

### DETAILED DESCRIPTION OF INVENTION

### Technical Problem

Under the above circumstances, the present inventors have conducted intensive studies to discover a prophylactic and therapeutic agent for proteinopathies by using a material that activates autophagy independently of mTOR, and as a result, have found that ligands binding to p62, more specifically, to the ZZ domain of p62, bind to LC3 and activates autophagy, resulting in the effective elimination of the pathological aggregates of proteins such as mutant huntingtin or alpha-synuclein, and thereby, it can be used for preventing, ameliorating or treating various proteinopathies. The present invention has been completed on the basis of such findings.

An object of the present invention is to provide a novel p62 ligand compound that induces activation and oligomerization of p62 protein.

In addition, an object of the present invention is to provide a method for delivering p62 and a misfolded protein to which p62 is bound, to autophagosome and finally delivering them to lysosome for elimination by using the above novel compound.

Another object of the present invention is to provide a method of increasing macroautophagy activity through p62 protein by using the novel compound.

In addition, an object of the present invention is to provide a pharmaceutical or food composition for eliminating a misfolded protein aggregate comprising the novel compound as an active ingredient.

Another object of the present invention is to provide a pharmaceutical or food composition for preventing, ameliorating or treating proteinopathies comprising the novel compound as an active ingredient.

### Solution to Problem

In order to solve the above object, the present invention provides a novel compound that acts as a ligand for p62 protein. Preferably, the novel p62 ligand according to the present invention binds to the ZZ domain of p62 protein.

In addition, the present invention provides a pharmaceutical composition for preventing or treating proteinopathies such as neurodegenerative diseases, or a health functional food for preventing or ameliorating misfolded protein-associated diseases, comprising a ligand binding to the ZZ domain of p62 protein as an active ingredient.

In addition, the present invention provides (1) a method for inducing p62 oligomerization and structural activation, (2) a method for increasing p62-LC3 binding, (3) a method for increasing the delivery of p62 to autophagosome, (4) a method for activating autophagy, and (5) a method for eliminating misfolded protein aggregates, which comprise treating a cell or p62 protein with a ligand that binds to the ZZ domain of p62.

The present invention provides a technology for eliminating misfolded protein aggregates, which are a causative factor of degenerative brain diseases, by activating p62 which delivers the misfolded protein aggregates directly to autophagosome.

The key technology of the present invention is to effectively eliminate misfolded protein aggregates, which cause degenerative brain diseases, by simultaneously activating p62 and autophagy.

The pharmacokinetics and key technologies of the present invention are summarized in FIG. 1.

Specifically, as shown in FIG. 1, major pathogenic proteins of proteinopathies such as mutant huntingtin and alpha-synuclein are converted to misfolded proteins which are insoluble in water, and then aggregated with each other and grow into oligomeric aggregates. These misfolded proteins grow further while acting as cytotoxic substances in neurons, and then grow into large oligomeric or fibrillar aggregates, eventually forming an inclusion body.

In the above process, endoplasmic reticulum chaperones (0) such as BiP produce a large amount of Nt-Arg through N-terminal argination (\textcircled{0}) by ATE1 R-transferase, and then arginylated BiP (R-BiP) is translocated into the cytoplasm and binds to the misfolded huntingtin or alpha-synuclein (\textcircled{3}). As a ligand, the Nt-Arg of R-BiP binds to the ZZ domain of p62. Due to the binding, the normally inactivated closed form of p62 is changed to an open form, leading to structural activation (\textcircled{4}), and as a result, PB1 and LC3-binding domains are exposed. This activation results in the formation of oligomers and high molecular weight aggregates due to disulfide bonds of p62 (\textcircled{5}), and increased binding to the autophagosome marker LC3 is finally delivered to autolysosome (\textcircled{6}). In addition, p62 bound with N-terminal arginine migrates to the endoplasmic reticulum membrane and activates PI3P-mediated autophagosome biogenesis (\textcircled{7}), thereby increasing intracellular autophagy (\textcircled{8}).

p62 is a first-in-class target for autophagy activation proposed by the present inventors (FIG. 1 and (\textcircled{8}). In addition, no previous studies proposing p62 as a drug target for the development of autophagy activation or the elimination of aggregates in misfolded protein-associated diseases such as degenerative brain diseases have been done.

Autophagy is a mechanism that acts to degrade or recycle cellular components that are unwanted or exhausted in cells, and it can act for the production of energy or metabolites to be used in biosynthetic process in conditions such as nutrient and energy deficiencies. The mechanism of autophagy is largely divided into macroautophagy, microautophagy, and chaperone-mediated autophagy, and it is divided into bulk autophagy and selective autophagy, depending on the purpose of degrading the intracellular substrate. Among them, selective autophagy and chaperone-mediated autophagy cause selective degradation of dysfunctional organelles or unwanted intracellular proteins. The development of new therapies for diseases based on the accumulation of malignant proteins and dysfunctional organelles by inducing selective autophagy is currently building a new paradigm.

The p62 protein is important for initiating the formation of autophagosome, which is a mediator in the mechanism for selective autophagy, and delivering the contents. It was observed that significant p62 activation of the novel p62 ligand according to the present invention induces p62 self-oligomerization. In addition, in light of the fact that autophagosome targeting of p62 through such self-oligomerization is increased, this demonstrates that the novel p62 ligands according to the present invention can induce the targeting and degradation of p62 protein by intracellular autophagy. These results mean that the novel p62 ligand compounds according to the present invention can be used as a more effective or supplemental alternative to existing anti-protein disease drugs.

PROteolysis Targeting Chimera (PROTAC) is a compound chimera of a ligand that recognizes a target protein and a ligand that recognizes an E3 ubiquitin enzyme. Since the paradigm of existing therapeutic agents for diseases is to inhibit protein enzyme, it is very important in developing a new therapeutic agent for the proteins that cannot be targeted with the existing therapeutic agent. From that point of view, PROTAC is an attractive new therapeutic development method by enabling selective degradation under ubiquitin-proteasome system with respect to proteins that cannot be targeted by a conventional enzyme inhibition method. However, currently, studies on PROTAC are limited only to the ubiquitin-proteasome system by utilizing only ligands that recognize the E3 ubiquitin enzyme, and thus has the folding problem associated with misfolded proteins in the aforementioned proteasome system. However, since the novel p62 ligand according to the present invention can induce intracellular autophagy as well as induce autophagosome targeting of cargo substrate proteins interacting with p62, it can provide a novel therapeutic agent that enables selective degradation under autophagy mechanisms of proteins that cannot be targeted by conventional enzyme inhibition methods.

### Advantageous Effects of Invention

The novel compound according to the present invention acts as a ligand binding to the ZZ domain of p62 protein, enhances the delivery of p62 to an autophagosome, activates autophagy, and eliminates misfolded protein aggregates, and therefore, is useful as a drug for preventing, ameliorating and treating various proteinopathies.

### BRIEF DESCRIPTION OF DRAWINGS

FIG. 1 is a schematic diagram illustrating that the proteins arginylated by N-terminal rule binds to the ZZ domain of p62, and degrades intracellular substances such as proteins through intracellular autophagy mechanism.

FIGS. 2a to 2d are immunoblot assay results showing the effects of increasing the p62 protein oligomerization activity of p62 ligand compounds (Example 2 (ATB10047), Example 1 (ATB10048), Example 3 (ATB10049), Example 5 (ATB10050), Example 4 (ATB10051), Example 7 (ATB10052), Example 6 (ATB10056), Example 8 (ATB10057), Example 9 (ATB10060), Example 10 (ATB10072), Example 11 (ATB10075), Example 12 (ATB10078), Example 13 (ATB10079), Example 14 (ATB10080), Example 15 (ATB10081), Example 16 (ATB10087), Example 17 (ATB0096), Example 18 (ATB10097), Example 19 (ATB10099), Example 20 (ATB10100)) according to the present invention.

FIGS. 3a to 3d are immunofluorescence staining assay results confirming that the p62 ligand compounds (Example 2 (ATB10047), Example 1 (ATB10048), Example 3 (ATB10049), Example 5 (ATB10050), Example 4 (ATB10051), Example 7 (ATB10052), Example 6 (ATB10056), Example 8 (ATB10057), Example 9 (ATB10060), Example 10 (ATB10072), Example 11 (ATB10075), Example 12 (ATB10078), Example 13 (ATB10079), Example 14 (ATB10080), Example 15 (ATB10081), Example 16 (ATB10087), Example 17 (ATB10096), Example 18 (ATB10097), Example 19 (ATB10099), and Example 20 (ATB10100)) according to the present invention allow the activation and oligomerization of p62 proteins, and then show the efficacy of delivering the proteins to be delivered to and degraded by autophagosome that is ubiquitinated in cells marked with FK2 and mediated by p62.

FIG. 4 is immunofluorescence staining assay results confirming that the p62 ligand compounds (Example 2 (ATB10047), Example 3 (ATB10049), Example 4 (ATB10051), Example 6 (ATB10056), Example 9 (ATB10060), Example 12 (ATB10078), Example 16 (ATB10087), Example 18 (ATB10097)) according to the present invention allow the activation and oligomerization of p62 protein, and show the efficacy of targeting them to the autophagosome essential for macroautophagy.

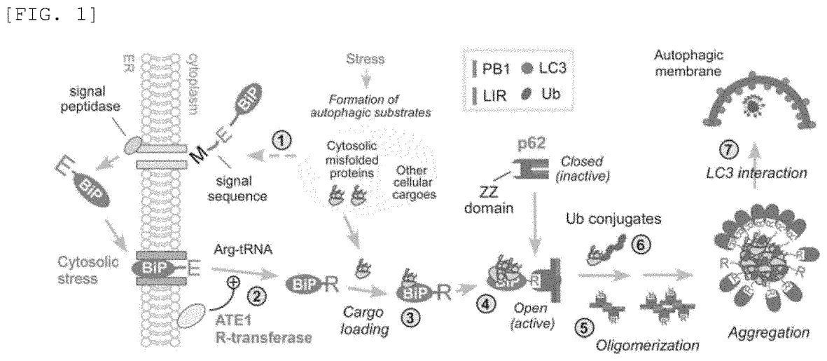

### BEST MODE OF EMBODIMENTS OF INVENTION

Hereinafter, the present invention will be described in detail.

The definition of each group used in the present specification is described in detail. Unless otherwise specified, each group has the following definitions.

In the present specification, examples of “halogen” comprise fluoro, chloro, bromo, and iodo.

In the present specification, “alkyl” refers to a linear or branched aliphatic saturated hydrocarbon group, preferably alkyl having 1 to 6 carbon atoms, more preferably alkyl having 1 to 4 carbon atoms. Examples of such alkyls comprise methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethyl butyl, 2,2-dimethylbutyl, 3,3-dimethylbutyl and 2-ethylbutyl.

In one aspect, the present invention relates to a compound represented by the following Chemical Formula 1, a pharmaceutically acceptable salt, stereoisomer, solvate, hydrate, or prodrug thereof.

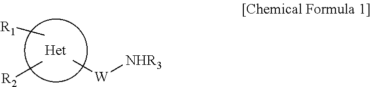

In Chemical Formula 1 above,

Het is a 4-10 membered heteroaryl or heterocyclyl comprising one or more heteroatoms selected from the group consisting of N, O and S;

R₁ and R₂ are each independently H, alkoxy having 1 to 4 carbon atoms, —NH— (CH₂)ₙ₁—R′, —O—(CH₂)ₙ₂—R′, or —(CH₂)ₙ₃—R′;

R′ is an aryl group having 6 to 10 carbon atoms;

W is a bond, —(CH₂)ₙ₄—, or —O—(CH₂)ₙ₅—CH(OH)—(CH₂)ₙ₆—;

n1, n2, n3, n4, n5 and n6 are each independently an integer of 0 to 3; preferably an integer of 1 or 2;

R₃ may be a substituted or unsubstituted alkylene group having 1 to 4 carbon atoms, or acyl group having 1 to 4 carbon atoms, and the substituent of the substituted acyl group having 1 to 4 carbon atoms or alkylene group having 1 to 4 carbon atoms is —OH, —NH₂ or —COOR″ (herein, R″ is H or an alkyl group having 1 to 3 carbon atoms).

Preferably, the Het group may be the 4 to 7 membered heteroaryl or 5 to 6 membered heteroaryl. More preferably, the Het group may be thiazolyl, thienyl, furanyl, pyridinyl or pyrimidinyl.

Preferably, the R₁ and R₂ may be each independently H, alkoxy having 1 to 3 carbon atoms, —NH—(CH₂)ₙ₁—R′, —O—(CH₂)ₙ₂—R′, or —(CH₂)ₙ₃—R′.

Preferably, R′ may be a phenyl group.

Preferably, n1, n2, n3, n4, n5 and n6 may be each independently an integer of 1 to 3, 0 to 2, 0 to 1, or 1 to 3.

More preferably, the R₁ and R₂ may be each independently H, methoxy,

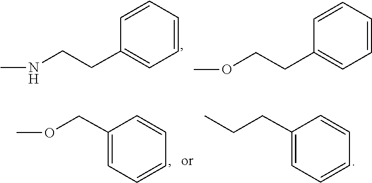

Preferably, W may be a bond, methylene, or —O—CH₂—CH(OH)—(CH₂)—.

Preferably, R₃ may be a substituted or unsubstituted methylene, ethylene or propylene, or a substituted or unsubstituted acyl group having 1 to 3 carbon atoms, and the substituent of the substituted acyl group having 1 to 3 carbon atoms or methylene, ethylene or propylene may be —OH, —NH₂, or —COOCH₃.

In a specific aspect, the compounds of the chemical formula 1 according to the present invention may be a compound selected from the group consisting of the compounds described in Examples 1 to 20 below:

TABLE1

| Example | | |
|---|---|---|
| No. | ID | CompoundName |
| Example | ATB10048 | N-((6-benzyloxy)pyridin-2- |
| 1 | | yl)methyl)-2-hydroxyacetamide |
| Example | ATB10047 | 2-(((6-(benzyloxy)pyridin-2- |
| 2 | | yl)methyl)amino)ethan-1-ol |
| Example | ATB10049 | N-((5-(benzyloxy)pyridin-2- |
| 3 | | yl)methyl)-2-hydroxyacetamide |
| Example | ATB10051 | N-((4-(benzyloxy)pyridin-2- |
| 4 | | yl)methyl)-2-hydroxyacetamide |
| Example | ATB10050 | 2-(((4-(benzyloxy)pyridin-2- |
| 5 | | yl)methyl)amino)ethan-l-ol |
| Example | ATB10056 | 1-((5-(benzyloxy)pyridin-2- |
| 6 | | yl)methyl)urea |
| Example | ATB10052 | N-((4,5-bis(benzyloxy)pyridin-2- |
| 7 | | yl)methyl)-2-hydroxyacetamide |
| Example | ATB10057 | 2-(((4,5-bis(benzyloxy)pyridin-2- |
| 8 | | yl)methyl)amino)ethan-l-ol |
| Example | ATB10060 | 2-(((5-(benzyloxy)pyrimidin-2- |
| 9 | | yl)methyl)amino)ethan-l-ol |
| Example | ATB10072 | (R)-1-((4-(benzyloxy)pyridin-2- |
| 10 | | yl)oxy)-3-((2- |
| | | hydroxyethyl)amino)propan-2-ol |
| Example | ATB10075 | (R)-1-((6-(benzyloxy)-5- |
| 11 | | methoxypyridin-2-yl)oxy)-3-((2- |
| | | hydroxyethyl)amino)propan-2-ol |
| Example | ATB10078 | Methyl(R)-3-((3-((4- |
| 12 | | (benzyloxy)pyridin-2-yl)oxy)-2- |
| | | hydroxypropyl)amino)propanoate |
| Example | ATB10079 | (R)-1-((5-(benzyloxy)pyridin-3- |
| 13 | | yl)oxy)-3-((2- |
| | | hydroxyethyl)amino)propan-2-ol |
| Example | ATB10080 | (R)-1-((6-(benzylamino)pyridin-2- |
| 14 | | yl)oxy)-3-((2- |
| | | hydroxyethyl)amino)propan-2-ol |
| Example | ATB10081 | (R)-1-((6-(benzyloxy)pyridin-2- |
| 15 | | yl)amino)-3-((2- |
| | | hydroxyethyl)amino)propan-2-ol |
| Example | ATB10087 | 2-(((5-phenethylfuran-2- |
| 16 | | yl)methyl)amino)ethan-1-ol |
| Example | ATB10096 | 2-(((5-(benzyloxy)thiophen-2- |
| 17 | | yl)methyl)amino)ethan-1-ol |
| Example | ATB10097 | 2-(((5-(benzyloxy)-furan-2- |
| 18 | | yl)methyl)amino)ethan-1-ol |
| Example | ATB10099 | 2-hydroxy-N-((5-phenethylfuran-2- |
| 19 | | yl)methyl)acetamide |
| Example | ATB10100 | ((5-phenethoxythiophen-2- |
| 20 | | yl)methyl)glycine |

Meanwhile, the compound of the present invention may exist in the form of a pharmaceutically acceptable salt. As the salt, an acid addition salt formed by a pharmaceutically acceptable free acid is useful. The term “pharmaceutically acceptable salt” of the present invention refers to any and all organic or inorganic addition salts of said compounds in which adverse effect caused by the salt does not impair the beneficial effect of the compound according to the present invention at a concentration exhibiting relatively non-toxic and non-harmful effective activity to a patient.

The acid addition salt is prepared by a common method, for example, by dissolving a compound in an excess amount of aqueous acid solution, and precipitating this salt using a water-miscible organic solvent such as methanol, ethanol, acetone or acetonitrile. An equimolar amount of a compound and an acid in water or alcohol (e.g., glycol monomethyl ether) can be heated, and subsequently, the resulting mixture can be dried by evaporating, or precipitated salts can be filtered under suction.

In this case, the free acid may be an organic acid and an inorganic acid. The inorganic acid may comprise, but is not limited to, hydrochloric acid, phosphoric acid, sulfuric acid, nitric acid, tartaric acid, etc. The organic acid may comprise, but is not limited to, methanesulfonic acid, p-toluenesulfonic acid, acetic acid, trifluoroacetic acid, maleic acid, succinic acid, oxalic acid, benzoic acid, tartaric acid, fumaric acid, manderic acid, propionic acid, citric acid, lactic acid, glycollic acid, gluconic acid, galacturonic acid, glutamic acid, glutaric acid, glucuronic acid, aspartic acid, ascorbic acid, carbonic acid, vanillic acid, hydroiodic acid, etc.

In addition, a pharmaceutically acceptable metal salt can be made using a base. An alkali metal salt or alkaline earth metal salt is obtained, for example, by dissolving a compound in an excess amount of alkali metal hydroxide or alkaline earth metal hydroxide solution, filtering the undissolved compound salt, and then evaporating the filtrate until dry. In this case, as the metal salt, it is particularly suitable for pharmaceutical use to prepare sodium, potassium, or calcium salt, but is not limited thereto. In addition, the corresponding silver salt can be obtained by reacting an alkali metal or alkaline earth metal salt with a proper silver salt (e.g., silver nitrate).

The pharmaceutically acceptable salt of the compound of the present invention, unless otherwise indicated, comprises a salt of acidic or a basic group, which may be present in the compounds represented by Chemical Formula 1 above. For example, the pharmaceutically acceptable salt may comprise sodium, calcium and potassium salt of hydroxy group, and other pharmaceutically acceptable salt of amino group, comprising hydrobromide, sulfate, hydrogen sulfate, phosphate, hydrogen phosphate, dihydrogen phosphate, acetate, succinate, citrate, tartrate, lactate, mandelate, methanesulfonate (mesylate) and p-toluenesulfonate (tosylate) salt, and the like. The salt may be prepared using a salt preparation method known in the art.

The salt of the compound of Chemical Formula 1 of the present invention is a pharmaceutically acceptable salt, and can be used without particular limitation as long as it is a salt of the compound of Chemical Formula 1 which can exhibit a pharmacological activity equivalent to that of the compound of Chemical Formula 1, for example, can prevent or treat neurodegenerative diseases by inducing autophagic degradation of intracellular neurodegenerative disease and tumor-associated proteins through a ligand of p62.

In addition, the compound represented by Chemical Formula 1 according to the present invention comprises, but is not limited thereto, not only a pharmaceutically acceptable salt thereof, but also a solvate such as a possible hydrate, and all possible stereoisomers that can be prepared therefrom. All stereoisomers of the present invention (e.g., those that may exist due to asymmetric carbons on various substituents), comprising enantiomeric form and diastereomeric form, are contemplated within the scope of the present invention. Individual stereoisomers of the compounds of the invention may be, for example, substantially free of other isomers (e.g., as a pure or substantially pure optical isomer having a certain activity), or, may be admixed, for example, as racemates or with all other, or other selected, stereoisomers. The chiral centers of the compounds of the present invention may have the S or R configuration as defined by the IUPAC 1974 Recommendations. The racemic form can be analyzed by physical methods, such as separation by chiral column chromatography, separation or crystallization of diastereomeric derivatives, or fractional shape crystallization. The individual optical isomers can be obtained from the racemates by any suitable method comprising, but not limited to, salt formation with an optically active acid followed by crystallization.

The solvate and stereoisomer of the compound represented by Chemical Formula 1 may be prepared from the compound using methods known in the art.

Furthermore, the compound represented by Chemical Formula 1 according to the present invention may be prepared in a crystalline form or in a non-crystalline form, and when prepared in a crystalline form, the compound may be optionally hydrated or solvated. In the present invention, the compound represented by Chemical Formula 1 may not only comprise a stoichiometric hydrate, but also comprise a compound comprising various amounts of water. The solvate of the compound represented by Chemical Formula 1 according to the present invention comprises both a stoichiometric solvate and a non-stoichiometric solvate.

In the preparation method of the present invention, as the reactants used in the above Reaction Schemes, commercially available compounds may be purchased and used as they are, or one or more reactions known in the art may be synthesized and used as they are or by appropriately being modified. For example, in consideration of the presence, type, and/or position of reactive functional groups and/or hetero elements contained in the skeletal structure, the reactants may be synthesized by performing one or more reactions in a series of order, but are not limited thereto.

The compound represented by Chemical Formula 1 according to the present invention is characterized by functioning as a ligand that binds to the ZZ domain of p62, and activating the function of p62. By activating the function of p62, the compound represented by Chemical Formula 1 according to the present invention can activate autophagy.

Accordingly, in another aspect, the present invention provides a pharmaceutical composition for autophagy activation, comprising the compound represented by Chemical Formula 1, a pharmaceutically acceptable salt, stereoisomer, hydrate, solvate, or prodrug thereof.

The compound represented by Chemical Formula 1 according to the present invention can eliminate aggregated proteins linked to misfolded protein aggregation-related diseases due to the activating action of autophagy. In addition, the compound is a p62 ligand, which binds to the ZZ domain of p62, and activates PB1 domain and LIR domain of the p62 protein, so that it induces p62 oligomerization and aggregates, and also increases autophagosome formation by inducing p62 aggregates. Through the above process, the misfolded protein aggregation is effectively eliminated. Such protein may be a main protein of proteinopathies, more preferably, one or more selected from the group consisting of prion protein, amyloid precursor protein (APP), alpha-synuclein, superoxide dismutase 1, tau, immunoglobulin, amyloid-A, transtyretin, beta 2-microglobulin, cystatin C, Apolipoproteine A1, TDP-43, islet amyloid polypeptide, ANF, gelsolin, insulin, lysozyme, fibrinogen, huntingtin, alpha-1-antitrypsin Z, crystallin, c9 open reading frame 72 (c9orf72), glial fibrillary acidic protein, cystic fibrosis transmembrane conductance regulator protein, rhodopsin and ataxin, and other proteins having Poly-Q stretch.

Accordingly, in still another aspect, the present invention provides a pharmaceutical composition for preventing or treating proteinopathies comprising the p62 ligand compound of Chemical Formula 1, a pharmaceutically acceptable salt, stereoisomer, hydrate, solvate, or prodrug thereof.

The term “aggregation” in accordance with the present invention refers to the formation of oligomeric or multimeric complexes of typically one or more types of proteins, which may be accompanied by the integration of additional biomolecules such as carbohydrates, nucleic acids and lipids, into the complexes. Such aggregated proteins may form deposits in specific tissues, more preferably in nerve tissues or brain tissues. The extent of aggregation depends on the particular disease.

The term “proteinopathy” or “disease linked to protein aggregation” as used herein, refers to those diseases which are characterized by the presence of aggregated proteins. Examples thereof comprise, but are not limited to, neurodegenerative diseases, anti-alpha-1 antitrypsin deficiency, keratopathy, retinitis pigmentosa, type 2 diabetes, and cystic fibrosis.

The neurodegenerative disease herein is preferably selected from the group consisting of Lyme borreliosis, Fatal familial insomnia, Creutzfeldt-Jakob Disease (CJD), multiple sclerosis (MS), dementia, Alzheimer disease, epilepsy, Parkinson's disease, stroke, Huntington's disease, Picks disease, amyotrophic lateral sclerosis (ALS), spinocerebellar ataxia, other poly-Q diseases, hereditary cerebral amyloid angiopathy, familial amyloid polyneuropathy, primary systemic amyloidosis (AL amyloidosis), reactive systemic amyloidosis (AA amyloidosis), injection-localized amyloidosis, beta-2 microglobulin amyloidosis, hereditary non-neuropathic amyloidosis, Alexander disease and Finnish hereditary systemic amyloidosis.

The dosage of the pharmaceutical composition of the present invention may vary with a broad range depending on the weight, age, gender, health condition of a patient, diet, administration period, administration method, excretion rate, and severity of disease. However, the effective dosage is usually about 1 ng to 10 mg/day, and particularly about 1 g to 1 mg/day for an adult (60 kg). Since the dosage is variable according to various conditions, it would be apparent to those skilled in the art that the dosage may be increased or decreased. Therefore, the dosage does not limit the scope of the present invention in any way. The number of administrations may be made once a day or divided into several times a day within a desired range, and the administration period is not particularly limited.

The term “treatment” of the present invention refers to all actions that alleviate or beneficially change the symptoms of various diseases linked to misfolded protein aggregation, such as cancer or neurodegenerative diseases by administration of the pharmaceutical composition of the present invention.

As described above, the compound of the present invention exhibits the effects of (1) inducing p62 oligomerization and structural activation, (2) increases p62-LC3 binding, and (3) increasing the delivery of p62 to autophagosomes, (4) activating autophagy, and finally (5) eliminating misfolded protein aggregates. Therefore, the pharmaceutical composition comprising this compound as an active ingredient can be used for preventing, ameliorating, or treating diseases linked to various misfolded protein aggregation.

For example, the composition of the present invention may further comprise a pharmaceutically acceptable carrier, diluents or excipients. The composition can be used in the various forms such as oral dosage forms of powders, granules, tablets, capsules, suspensions, emulsions, syrups, and aerosols, and injections of a sterile injectable solutions, which are formulated by the conventional method according to the purpose of each of the intended use. The composition can be administered through various routes comprising oral administration or intravenous, intraperitoneal, subcutaneous, rectal, and topical administration. Examples of suitable carriers, excipients or diluents which can be comprised in such compositions may comprise lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methyl hydroxy benzoate, propylhydroxy benzoate, talc, magnesium stearate, mineral oil, and the like. In addition, the composition of the present invention may further comprise fillers, anti-coagulants, lubricants, humectants, fragrances, emulsifiers, preservatives, and the like.

A solid formulation for oral administration comprise tablets, pills, powders, granules, capsules, and the like, and such solid formulation is formulated by mixing the composition with one or more excipients, such as starch, calcium carbonate, sucrose, lactose, gelatin, and the like. In addition, lubricants such as magnesium stearate and talc may be used in addition to simple excipients.

A liquid formulation for oral administration can be illustrated as suspensions, solutions, emulsions, syrups, and the like, and can comprise various excipients, such as humectants, sweeteners, fragrances, preservatives and the like, in addition to water and liquid paraffin, which are commonly used simple diluents.

A formation for parenteral administration comprise sterilized aqueous solutions, non-aqueous solutions, suspension agents, emulsion agents, lyophilizing agents, and suppository agents. Non-aqueous solvent and suspending agent may comprise propylene glycol, polyethylene glycol, vegetable oil such as olive oil, and injectable esters such as ethyl oleate. As a substrate for the suppository agent, Withepsol, Macrogol, and Tween61, Cacao butter, laurin paper, glycerogelatin, and the like can be used. Meanwhile, the injections may comprise conventional additives such as solubilizing agents, isotonic agents, suspending agents, emulsifiers, stabilizers, or preservatives.

The formulation may be prepared by a conventional mixing, granulating or coating method, and comprises an active ingredient in an amount effective for medical treatment, specifically preventing, ameliorating or treating diseases linked to misfolded protein aggregation.

In this case, the composition of the present invention is administered in a pharmaceutically effective amount. The term “pharmaceutically effective amount” of the present invention refers to an amount which is sufficient to treat a disease at a reasonable benefit/risk ratio applicable to any medical treatment, and also which is enough to not cause side effects. The level of effective amount can be determined depending on patient's health condition, disease type, severity of the disease, activity of the drug, sensitivity on the drug, administration method, administration time, administration route, excretion rate, treatment duration, combination, factors comprising other medicines used at the same time and other factors well-known in the medical field. The composition of the present invention may be administered as an individual therapeutic agent or in combination with other therapeutic agents, and it may be administered sequentially or simultaneously with a conventional therapeutic agent, and once or multiple times. It is important to administer the minimum amount which can provide the maximum effect without side effects in consideration of all the above factors, which can be easily determined by a person skilled in the art.

For example, the dosage may be increased or decreased depending on administration route, the severity of a disease, gender, weight, age, and the like, and the scope of the present invention is not limited by the aforementioned dosage in any way.

A preferred dose of the compound according to the present invention may be varied according to the condition and weight of a patient, the severity of a disease, the type of a drug, and the route and duration of administration, but it may be appropriately selected by a person skilled in the art.

In still another aspect, the present invention provides a method for increasing the degradation of misfolded protein aggregates, a method for activating autophagy, or a method for preventing, ameliorating or treating proteinopathies, comprising administering a p62 ligand compound of Chemical Formula 1 of the present invention, or a pharmaceutical composition comprising the same to a subject in need thereof.

The term “subject” as used herein refers to all animals comprising human, monkeys, cows, horses, sheep, pigs, chickens, turkeys, quail, cat, dog, mouse, rat, rabbit, or guinea pig, which have the potential of metastasis and invasion of cancer, or cancer already metastasized and invaded, or have diseases linked to misfolded protein aggregation. The diseases linked to misfolded protein aggregation can be effectively prevented, ameliorated or treated by administrating the pharmaceutical composition of the present invention to the subject. In addition, since the pharmaceutical composition of the present invention functions as a p62 ligand to activate autophagy, eliminates aggregates of cancer-inducing proteins or misfolded proteins due to the autophagy activation, and thus exhibits a prophylactic or therapeutic effect of diseases linked to these aggregated proteins, it can exhibit synergistic effects by administration in combination with existing therapeutic agent.

The term “administration” of the present invention refers to introduction of a predetermined substance to a patient in certain appropriate method, and the route of administration of the composition of the present invention can be administered through any general route as long as it can reach a target tissue. Intraperitoneal administration, intravenous administration, intramuscular administration, subcutaneous administration, intradermal administration, oral administration, topical administration, intranasal administration, intrapulmonary administration, and intrarectal administration, but the route is not limited thereto. In addition, the pharmaceutical composition of the present invention may be administered using any device capable of delivering the active ingredients to target cells. Preferred administration modes and formulations are an intravenous injection, a subcutaneous injection, an intradermal injection, an intramuscular injection, drip injections, and the like. Injectable formulations may be prepared using aqueous solutions such as Ringer's solution, saline, and non-aqueous solutions, such as vegetable oils, high fatty acid esters (e.g., ethyl oleic acid, etc.), alcohols (e.g., ethanol, benzyl alcohol, propylene glycol, glycerin, etc.). The injectable formulations may comprise pharmaceutical carriers such as stabilizer for preventing deterioration (e.g., ascorbic acid, sodium hydrogen sulfite, sodium pyrosulfite, BHA, tocopherol, EDTA, etc.), an emulsifier, a buffering agent for pH control, and a preservative for inhibiting microbial growth (e.g., phenylmercuric nitrate, thimerosal, benzalkonium chloride, phenol, cresol, benzyl alcohol, etc.).

In still another aspect, the present invention provides a food composition for preventing or ameliorating proteinopathies comprising a p62 ligand compound of Chemical Formula 1, a pharmaceutically acceptable salt, stereoisomer, hydrate, solvate, or prodrug thereof. The food composition is a health functional food and it can be used through formulation itself, or be comprised in other health functional foods as an additive of health functional food. The health functional food refers to a food that has body modulating function such as preventing or ameliorating disease, biodefense, immunity, recovery of convalescence, aging inhibition, etc., and it should be harmless to human body when taking in a long time. The mixing amount of active ingredients may be appropriately decided depending on purpose of use (prevention, health or therapeutic treatment).

There is no particular limitation on the type of the food. Examples of foods to which the above substances can be added comprise meat, sausage, bread, chocolate, candy, snacks, snack, pizza, ramen, other noodles, chewing gum, dairy products comprising ice cream, various soups, beverages, tea, health drinks, alcoholic beverages and vitamin complexes, and all health functional foods in the common sense.

The food composition of the present invention may comprise common ingredients used in the preparation of food or food additives, specifically, a flavoring agent; a natural sweetener such as, monosaccharides like glucose and fructose, disaccharides like as maltose and sucrose, and dextrin, cyclodextrin as a natural carbohydrate, or a synthetic sweetener such as saccharin and aspartame; a nutrient; vitamin; electrolyte; a coloring agent; an organic acid; a protective colloid viscosity agent; pH regulator; a stabilizer; a preservative; glycerin; alcohol; a carbonating agent used in carbonated drinks, etc.

### Embodiments of Invention

### Example

The present invention will be described in more detail with reference to the following examples. These examples are for explaining the present invention more specifically, and the scope of the present invention is not limited to these examples.

Among the compounds of Chemical Formula 1, the compounds of Examples 1-20 were prepared according to the following method.

In the case of the starting materials for synthesizing the compounds of the present invention, various synthesis methods have been known, and if available on the market, the starting materials may be purchased from the suppliers. Reagent suppliers comprise companies such as Aldrich, Sigma, TCI, Wako, Kanto, Fluorchem, Acros, Alfa, and Fluka, but are not limited thereto.

The compounds of the present invention can be prepared from readily available starting materials using the following general methods and procedures. As for typical or preferred process conditions (i.e., reaction temperature, time, molar ratio of reactants, solvent, pressure) and the like, other process conditions may be used unless otherwise stated. The optimum reaction state may vary depending on the specific reactant or solvent used, but such condition can be determined by a person skilled in the art by conventional optimization procedures.

Hereinafter, the manufacturing method of Examples 1 to 20 will be described.

### Preparation Example 1) the Compounds of Examples 1 and 2 were Synthesized by the Method Disclosed in Reaction Scheme 1 Below

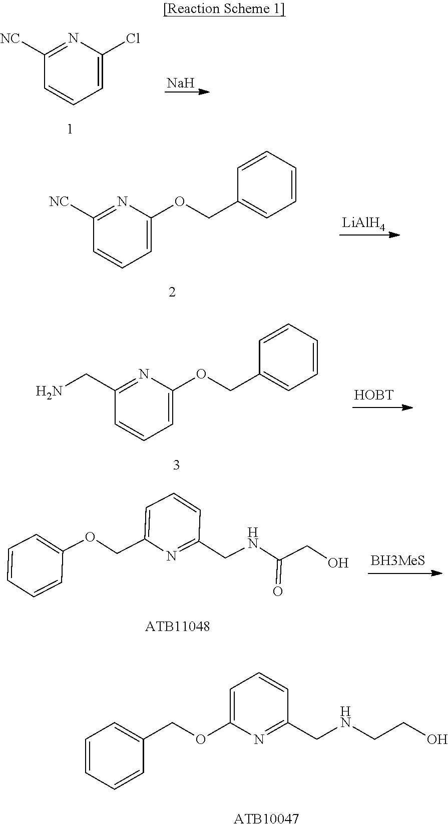

### Example 1: Preparation of N-((6-benzyloxy)pyridin-2-yl)methyl)-2-hydroxyacetamide (ATB10048)

Step 1) Synthesis of 6-(benzyloxy)picolinonitrile (2): After 6-chloropicolino nitrile (1, 5.0 g, 36.2 mmol) and benzyl alcohol (7.8 g, 72.2 mmol) were dissolved in dimethylformamide (DMF, 50.0 ml), NaH (1.88 g, 47.0 mmol) was slowly added to the reaction solution, and stirred at room temperature for 12 hours. After the reaction solution was put in cold water, the resulting solid was filtered under reduced pressure and washed with water. The resulting solid was dried to obtain 6-(benzyloxy)picolinonitrile (2, 6.4 g) in the form of a yellow solid. ¹H NMR (400 MHz, DMSO_d₆: δ 7.93-7.95 (m, 1H), 7.66-7.67 (m, 1H), 7.34-7.50 (m, 5H), 7.24-7.26 (m, 1H), 5.37 (s, 2H).

Step 2) Synthesis of (6-(benzyloxy)pyridin-2-yl)methanamine (3): After 6-(benzyloxy) picolinonitrile (2, 3.5 g, 16.7 mmol) was dissolved in tetrahydrofuran (THF, 35.0 ml), it was cooled to −10° C., and LiAlH₄ (1.27 g, 33.4 mmol) was added. After the reaction solution was stirred at −10° C. for 2 hours, water (2.86 ml) was added to complete the reaction. After the reaction solution was cooled to −20° C., 15% NaOH aqueous solution (0.95 ml) was added and further stirred at room temperature for 30 minutes. After the reaction solution was filtered through Celite, the resulting filtrate was concentrated under reduced pressure to obtain (6-(benzyloxy)pyridin-2-yl)methanamine (3, 2.7 g) in the form of a yellow oil. ESI-MS Calcd m/z for C₁₃H₁₄N₂O [M]⁺ 214.27 Found 215.1.

Step 3) Synthesis of N-((6-benzyloxy)pyridin-2-yl)methyl)-2-hydroxyacetamide (ATB10048): After 2-hydroxyacetic acid (1.37 g, 18.0 mmol), hydroxybenzotriazole (HOBT, 2.43 g, 18.0 mmol), 1-ethyl-3-(3-dimethylaminopropyl)carbodiimide (EDCI, 3.45 g, 18.0 mmol) were sequentially added in a flask containing (6-(benzyloxy)pyridin-2-yl)methanamine (3, 3.5 g, 16.4 mmol), and dissolved in dimethylformamide (DMF, 35.0 ml), diisopropylethylamine (DIEA, 5.27 g, 40.9 mmol) was added and stirred at room temperature for 14 hours. When the reaction was complete, cold water was added to the reaction solution, extracted with ethyl acetate (EA), and an organic layer was washed once more with brine. The organic layer was dehydrated with sodium sulfate (Na₂SO₄), filtered under reduced pressure, and the filtrate was concentrated under reduced pressure. The reaction concentrate was purified by silica gel column chromatography to synthesize N-((6-benzyloxy)pyridin-2-yl)methyl)-2 hydroxyacetamide (ATB10048, 1.15 g) in the form of a pure white solid. ¹H NMR (400 MHz, CDCl₃): δ 7.54-7.58 (m, 1H), 7.26-7.50 (m, 5H), 6.82-6.84 (m, 1H), 6.70-6.73 (m, 1H), 5.39 (s, 2H), 4.51-4.53 (m, 2H), 4.13-4.14 (m, 2H), 2.44 (m, 1H)., ESI-MS Calcd m/z for C₁₅H₁₆N₂O₃ [M]⁺ 273.10 Found 272.30.

### Example 2: Preparation of 2-(((6-(benzyloxy)pyridin-2-yl)methyl)amino)ethan-1-ol (ATB10047)

After N-((6-benzyloxy)pyridin-2-yl)methyl)-2-hydroxyacetamide (ATB10048, 900 mg, 3.3 mmol) was dissolved in tetrahydrofuran (THF, 10 ml), borane dimethyl sulfide (BH₃Me₂S, 0.83 ml, 8.27 mmol, 10 M) was added and stirred at 55° C. for 12 hours. After the reaction solution was cooled to room temperature, methanol (20.0 ml) was added to the reaction solution to complete the reaction, followed by concentration under reduced pressure. The concentrated solution was purified by high-resolution liquid chromatography to synthesize 2-(((6-(benzyloxy)pyridin-2-yl)methyl)amino)ethan-1-ol (ATB10047, 70 mg) in the form of a colorless oil. ¹H NMR (400 MHz, DMSO_d₆: δ 8.24 (s, 1H), 7.68-7.72 (m, 1H), 7.32-7.47 (m, 5H), 7.02-7.04 (m, 1H), 6.74-6.76 (m, 1H), 5.36 (s, 2H), 3.86 (s, 2H), 3.52-3.55 (m, 2H), 2.70-2.73 (m, 2H)., ESI-MS Calcd m/z for C₁₅H₁₈N₂O₂ [M]⁺ 259.00 Found 258.32.

### Preparation Example 2) the Compound of Example 3 was Synthesized by the Method Disclosed in Reaction Scheme 2 Below

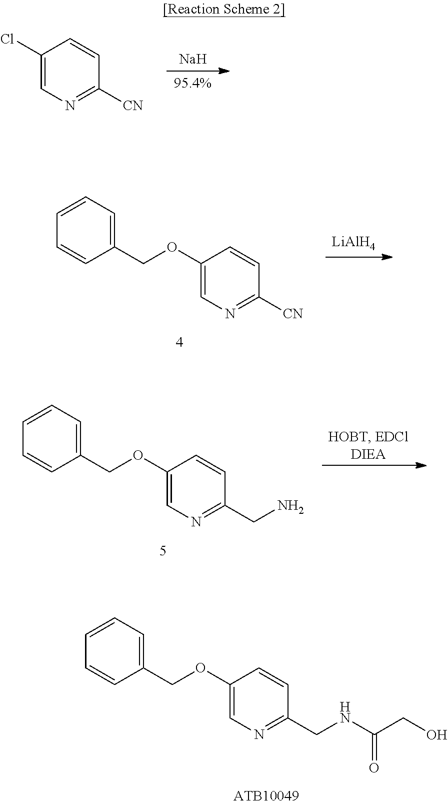

### Example 3: Preparation of N-((5-(benzyloxy)pyridin-2-yl)methyl)-2-hydroxyacetamide (ATB10049)

Step 1) Synthesis of 5-(benzyloxy)picolinonitrile (4): After 5-chloropicolino nitrile (10 g, 72.5 mmol) and benzyl alcohol (11.7 g, 109 mmol) were dissolved in dimethylformamide (DMF, 100.0 ml), NaH (4.35 g, 109 mmol) was slowly added to the reaction solution and stirred at room temperature for 12 hours. After the reaction solution was put in cold water, the resulting solid was filtered under reduced pressure and washed with water. The resulting solid was dried to obtain 5-(benzyloxy)picolinonitrile (4, 14.5 g) in the form of a white solid. ESI-MS Calcd m/z for C₁₃H₁₀N₂O [M]⁺ 210.24 Found 211.

Step 2) Synthesis of (5-(benzyloxy)pyridin-2-yl)methanamine (5): After 5-(benzyloxy) picolinonitrile (4, 14.5 g, 69.0 mmol) was dissolved in tetrahydrofuran (THF, 200 ml), the reaction solution was cooled to −10° C., and LiAlH₄ (3.94 g, 104 mmol) was added. After the reaction solution was stirred at −10° C. for 2 hours, water (15.76 ml) was added to complete the reaction. After the reaction solution was cooled to −20° C., 15% NaOH aqueous solution (3.94 ml) was added and further stirred at room temperature for 30 minutes. After the reaction solution was filtered through Celite, the resulting filtrate was concentrated under reduced pressure to obtain (5-(benzyloxy)pyridin-2-yl)methanamine (5, 2.50 g) in the form of a yellow oil. ¹H NMR (400 MHz, CDCl₃): δ 8.31-8.32 (m, 1H), 7.32-7.43 (m, 5H), 7.18-7.24 (m, 2H), 5.10 (s, 2H), 3.91 (s, 2H).

Step 3) Synthesis of N-((5-(benzyloxy)pyridin-2-yl)methyl)-2-hydroxyacetamide (ATB10049): After 2-hydroxyacetic acid (355 mg, 4.67 mmol), hydroxybenzotriazole (HOBT, 631 mg, 4.67 mmol), 1-ethyl-3-(3-dimethylaminopropyl)carbodiimide (EDCI, 893 mg, 4.68 mmol) were sequentially added to a flask containing (5-(benzyloxy)pyridin-2-yl)methanamine (5, 1 g, 4.67 mmol), the reaction solution was dissolved in dimethylformamide (DMF, 15 ml). Then, diisopropylethylamine (DIEA, 1.51 g, 11.7 mmol) was added and stirred at room temperature for 12 hours. When the reaction is completed, cold water was added to the reactant, extracted with ethyl acetate (EA), and an organic layer was washed once more with brine. The organic layer was dehydrated with sodium sulfate (Na₂SO₄), filtered under reduced pressure, and the filtrate was concentrated under reduced pressure. The reaction concentrate was purified by silica gel column chromatography to synthesize N-((5-benzyloxy)pyridin-2-yl)methyl)-2-hydroxyacetamide (ATB10049, 750 mg) in the form of a pure white solid. ¹H NMR (400 MHz, CDCl₃) δ (ppm) 3.54 (s, 1H), 4.17 (s, 2H), 4.54 (d, J=4 Hz, 2H), 5.09 (s, 2H), 7.23 (m, 2H), 7.40 (m, 5H), 8.27 (s, 1H)., ESI-MS Calcd m/z for C₁₅H₁₆N₂O₃ [M]⁺ 273.00 Found 272.30.

### Preparation Example 3) the Compounds of Examples 4 and 5 were Synthesized by the Method Disclosed in Reaction Scheme 3 Below

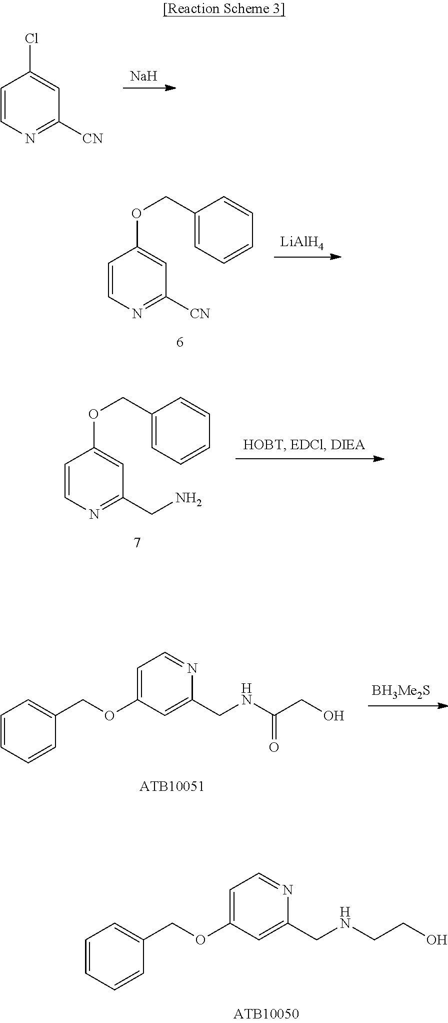

### Example 4: Preparation of N-((4-(benzyloxy)pyridin-2-yl)methyl)-2-hydroxyacetamide (ATB10051)

Step 1) Synthesis of 4-(benzyloxy)picolinonitrile (6): After 4-chloropicolinonitrile (5 g, 36.2 mmol) and benzyl alcohol (5.87 g, 54.3 mmol) were dissolved in dimethylformamide (DMF, 50.0 ml), NaH (2.17 g, 54.3 mmol) was slowly added to the reaction solution and stirred at room temperature for 12 hours. After the reaction solution was put in cold water, the resulting solid was filtered under reduced pressure and washed with water. The resulting solid was dried to obtain 4-(benzyloxy)picolinonitrile (6, 6.5 g) in the form of a yellow solid. ¹H NMR (400 MHz, CDCl₃): δ 8.50-8.51 (m, 1H), 7.38-7.43 (m, 5H), 7.26-7.28 (m, 1H), 7.05-7.07 (m, 1H), 5.16 (s, 2H).

Step 2) Synthesis of (4-(benzyloxy)pyridin-2-yl)methanamine (7): After 4-(benzyloxy)picolinonitrile (6, 5.0 g, 23.8 mmol) was dissolved in tetrahydrofuran (THF, 80 ml), the reaction solution was cooled to −10° C., and LiAlH₄ (1.36 g, 35.7 mmol) was added. After the reaction solution was stirred at −10° C. for 2 hours, water (4.08 ml) was added to complete the reaction. After the reaction solution was cooled to −20° C., 15% NaOH aqueous solution (1.36 ml) was added, and stirred at room temperature for minutes. After the reactant solution was filtered through Celite, the resulting filtrate was concentrated under reduced pressure to obtain (4-(benzyloxy)pyridin-2-yl)methanamine (7, 2.50 g) in the form of a yellow oil. ESI-MS Calcd m/z for C₁₃H₁₄N₂O [M]⁺ 214.27 Found 215.3.

Step 3) Synthesis of N-((4-(benzyloxy)pyridin-2-yl)methyl)-2-hydroxyacetamide (ATB10051): After 2-hydroxyacetic acid (887 mg, 11.7 mmol), hydroxybenzotriazole (HOBT, 1.58 g, 11.7 mmol), 1-ethyl-3-(3-dimethyl aminopropyl) carbodiimide (EDCI, 2.23 g, 11.7 mmol) were sequentially added to a flask containing (4-(benzyloxy)pyridin-2-yl)methanamine (2.50 g, 11.7 mmol), and dissolved in dimethylformamide (DMF, 30 ml), diisopropylethylamine (DIEA, 2.50 g, 11.7 mmol) was added and stirred at room temperature for 12 hours. When the reaction is complete, cold water was added to the reaction solution, extracted with ethyl acetate (EA), and an organic layer was washed once more with brine. The organic layer was dehydrated with sodium sulfate (Na₂SO₄), filtered under reduced pressure, and the filtrate was concentrated under reduced pressure. The reaction concentrate was purified by column chromatography to synthesize N-((4-benzyloxy)pyridin-2-yl)methyl)-2-hydroxyacetamide (ATB10051, 800 mg) in the form of a pure white solid. ¹H NMR (400 MHz, CDCl₃) δ (ppm) 4.18 (s, 2H), 4.55 (d, J=4 Hz, 2H), 5.10 (s, 2H), 6.80 (m, 1H), 6.87 (s, 1H), 7.38 (m, 5H), 7.60 (b, 1H), 8.31 (d, J=8 Hz, 1H)., ESI-MS Calcd m/z for C₁₅H₁₈N₂O₂ [M]⁺ 273.00 Found 272.3.

### Example 5: Preparation of 2-(((4-(benzyloxy)pyridin-2-yl)methyl)amino)ethan-1-ol (ATB10050)

After N-((4-benzyloxy)pyridin-2-yl)methyl)-2-hydroxyacetamide (ATB10051, 750 mg, 2.76 mmol) was dissolved in tetrahydrofuran (THF, 10 ml), borane dimethyl sulfide (BH₃Me₂S, 0.69 ml, 6.9 mmol, 10 mol/L) was added and stirred at 55° C. for 10 hours. After the reaction solution was cooled to room temperature, methanol (20.0 ml) was added to the reaction solution to complete the reaction, followed by concentration under reduced pressure. The concentrated solution was purified by high-resolution liquid chromatography to synthesize 2-(((4-(benzyloxy)pyridin-2-yl)methyl)amino)ethan-1-ol (ATB10050, 75 mg) in the form of a colorless oil. ¹H NMR (400 MHz, CDCl₃) δ (ppm) 2.84 (t, J=4 Hz, 2H), 3.65 (t, J=4 Hz, 2H), 3.89 (s, 2H), 5.11 (s, 2H), 6.77 (dd, J=4 Hz and 2.4 Hz, 1H), 6.88 (d, J=2.4 Hz, 1H), 7.38 (m, 6H), 8.37 (d, J=4 Hz, 1H)., ESI-MS Calcd m/z for C₁₅H₁₈N₂O₂ [M]⁺ 259.00 Found 258.32.

### Preparation Example 4) the Compound of Example 6 was Synthesized by the Method Disclosed in Reaction Scheme 4 Below

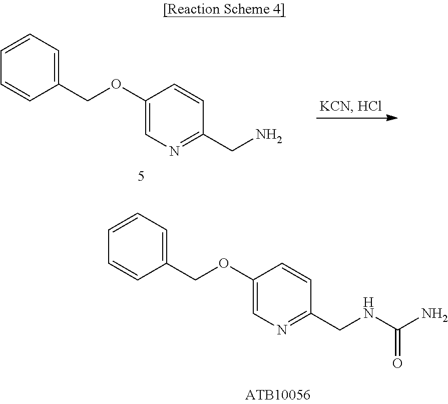 [SMILES: NCc1ccc(OCc2ccccc2)cn1 ; NC(=O)NCc1ccc(OCc2ccccc2)cn1]

### Example 6: Preparation of 1-((5-(benzyloxy)pyridin-2-yl)methyl)urea (ATB10056)

After (5-(benzyloxy)pyridin-2-yl)methanamine (5, 1 g, 4.76 mmol) and potassium cyanide (KCN, 3.86 g, 47.6 mmol) were dissolved in purified water (15 ml) and conc. HCl (4 ml), the reaction solution was stirred at 75° C. for 12 hours. After the reaction solution was cooled, it was added in ice water and extracted with ethyl acetate (EA). The extracted organic layer was dehydrated with sodium sulfate (Na₂SO₄), filtered and concentrated. The concentrated reaction solution was purified using high-resolution liquid chromatography to synthesize 1-((5-(benzyloxy)pyridin-2-yl)methyl) urea (ATB10056, 200 mg) in the form of a white solid. ¹H NMR (400 MHz, DMSO-d₆) δ (ppm) 4.19 (d, J=6 Hz, 2H), 5.16 (s, 2H), 5.58 (s, 2H), 6.45 (t, J=6 Hz, 1H), 7.21 (d, J=8.4 Hz, 1H), 7.31-7.46 (m, 6H), 6.25 (d, J=2.8 Hz, 1H)., Mass Calcd.: 257; MS Found: 258 [MS+1].

### Preparation Example 5) the Compounds of Examples 7 and 8 were Synthesized by the Method Disclosed in Reaction Scheme 5 Below

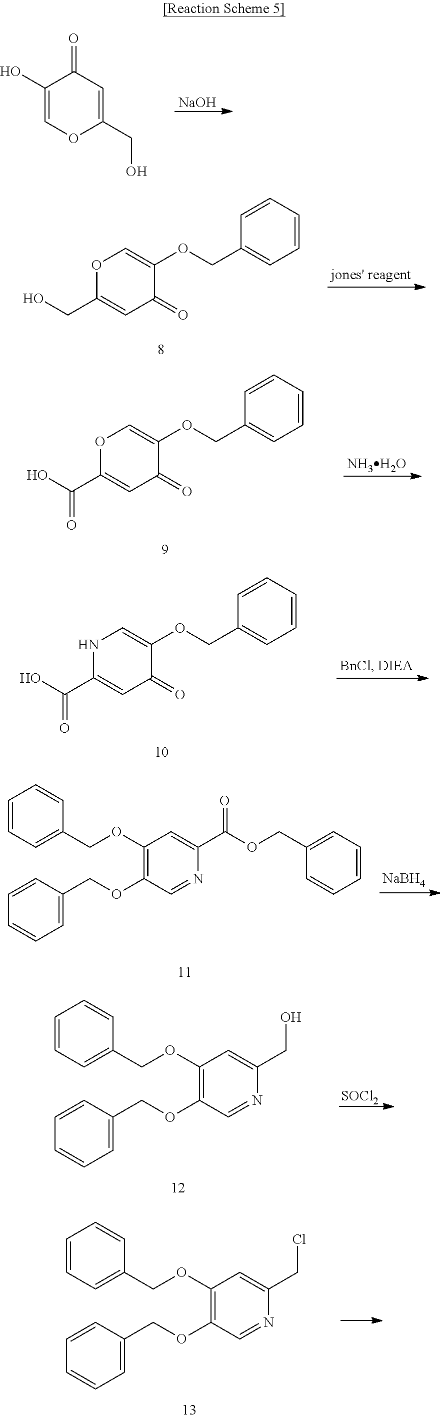

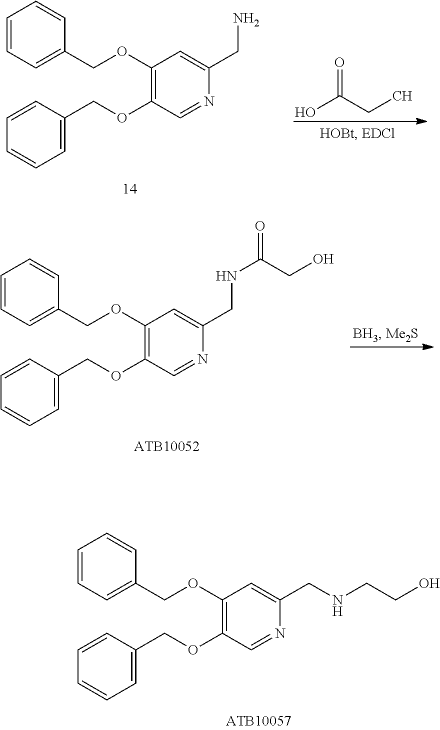

### Example 7: Preparation of N-((4,5-bis(benzyloxy)pyridin-2-yl)methyl)-2-hydroxyacetamide (ATB10052)

Step 1) synthesis of 5-(benzyloxy)-2-(hydroxymethyl)-4 H-pyran-4-one (8): After 5-hydroxy-2-(hydroxymethyl)-4H-pyran-4-one (kojic acid, 20.0 g, 141 mmol) was dissolved in methanol (200 ml), an aqueous solution in which NaOH (6.20 g, 155 mmol) was dissolved in water (20 ml) was added to the reaction solution, and benzyl chloride (27.6 g, 219 mmol) was slowly added dropwise to the reaction solution. The reaction solution was stirred at 65° C. for 12 hours, then cooled to room temperature, and added into water to form a solid. The reaction solution was filtered under reduced pressure, and then the resulting solid was dried to synthesize a white, 5-(benzyloxy)-2-(hydroxymethyl)-4 H-pyran-4-one (8, 25.0 g, 76.5% yield). ¹H NMR (400 MHz, DMSO_d₆): δ 8.17 (s, 1H), 7.40-7.42 (m, 5H), 6.32 (s, 1 H), 5.67-5.71 (m, 1H), 4.94 (s, 2H), 4.29-4.30 (m, 2H).

Step 2) synthesis of 5-(benzyloxy)-4-oxo-4H-pyran-2-carboxylic acid (9): After 5-(benzyloxy)-2-(hydroxymethyl)-4H-pyran-4-one (8, 18.0 g, 77.5 mmol) was dissolved in acetone (400 ml), Jone's reagent (2.5 M, 80.0 ml) was added and stirred at room temperature for 16 hours, followed by filtration. After the filtered solution was concentrated under reduced pressure, water was added and the resulting solid was filtered under reduced pressure, washed with water and dried to synthesize 5-(benzyloxy)-4-oxo-4H-pyran-2-carboxyl acid (9, 15.0 g, 78.9% yield) in the form of a white solid. ¹H NMR (400 MHz, DMSO_d₆): δ 8.37 (s, 1H), 7.37-7.45 (m, 5H), 6.94 (s, 1H), 4.98 (m, 2H).

Step 3) Synthesis of 5-(benzyloxy)-4-oxo-1,4-dihydropyridine-2-carboxylic acid (10): After 5-(benzyloxy)-4-oxo-4H-pyran-2-carboxylic acid (9, 15.0 g, 61.0 mmol) was dissolved in aqueous ammonia (200 ml), the reaction solution was reacted in a high-temperature sterilization reactor at 80° C. for 12 hours. HCl was added to the reaction solution, acidified to pH 3, and then, the resulting solid was filtered under reduced pressure. Then, this was washed with water and dried to synthesize a white, 5-(benzyloxy)-4-oxo-1,4-dihydropyridine-2-carboxylic acid (10, 12.0 g, 80% yield). Mass Calcd.: 245.2; MS Found: 246 [MS+1].

Step 4) Synthesis of benzyl 4,5-bis(benzyloxy)picolinate (11): After 5-(benzyloxy)-4-oxo-1,4-dihydropyridine-2-carboxylic acid (10, 12.0 g, 49.0 mmol) was dissolved in dimethylformamide (DMF, 120 ml), diisopropylethylamine (DIEA, 12.6 g, 98.0 mmol) was added, and then benzyl chloride (7.41 g, 58.8 mmol) was added. The reaction solution was stirred at 80° C. for 12 hours, cooled to room temperature, and stirred in cold water. The resulting solid was filtered under reduced pressure and dried to synthesize a white, benzyl 4,5-bis(benzyloxy)picolinate (11, 12.0 g, 57.7% yield). Mass Calcd.: 425.4; MS Found: 426 [MS+1].

Step 5) Synthesis of (4,5-bis(benzyloxy)pyridin-2-yl)methanol (12): After benzyl 4,5-bis(benzyloxy)picolinate (11, 10.0 g, 23.5 mmol) was dissolved in methanol, it was cooled to 0° C., and then NaBH₄ (1.15 g, 30.5 mmol) and CaCl₂ (3.91 g, 0.12 mmol) were slowly added in sequence. After the reaction solution was stirred at room temperature for 2 hours, it was added to cold water and stirred. The resulting solid was filtered under reduced pressure and dried to synthesize a white, (4,5-bis(benzyloxy)pyridin-2-yl)methanol (12, 7.0 g, 92.7% yield). Mass Calcd.: 321.38; MS Found: 322 [MS+1].

Step 6) Synthesis of 4,5-bis(benzyloxy)-2-(chloromethyl)pyridine (13): After (4,5-bis(benzyloxy)pyridin-2-yl)methanol (12, 7.0 g, 21.8 mmol) was dissolved in dichloromethane (DCM, 100 ml), SOCl₂ (3.89 g, 32.7 mmol) was added and stirred at room temperature for 8 hours. After the reaction solution was concentrated under reduced pressure, the solid obtained by filtration was washed with ethyl acetate. Subsequently, it was dried to synthesize 4,5-bis(benzyloxy)-2-(chloromethyl)pyridine (13, 5.0 g, 67.7% yield) in the form of a white solid. Mass Calcd.: 339.82; MS Found: 341 [MS+2].

Step 7) Synthesis of (4,5-bis(benzyloxy)pyridin-2-yl)methanamine (14): After 4,5-bis(benzyloxy)-2-(chloromethyl)pyridine (13, 5.0 g, 14.7 mmol) was dissolved in dichloromethane (DCM, 5 ml), hexamethylenetetramine (6.19 g, 44.2 mmol) was added to the reaction solution, and stirred at 45° C. for 12 hours, followed by concentration under reduced pressure. Methanol (50 ml) was added to the concentrated reaction solution to dissolve, and then conc. HCl (2 ml) was added and further stirred at 45° C. for 2 hours. The reaction solution was filtered under reduced pressure and the obtained solid was dried to synthesize (4,5-bis(benzyloxy)pyridin-2-yl)methanamine (14, 4 g, 84.7% yield) in the form of a light gray. ¹H NMR (400 MHz, DMSO_d₆): δ (ppm) 7.33-7.52 (m, 14H), 5.28-5.33 (m, 6H).

Step 8) Synthesis of N-((4,5-bis(benzyloxy)pyridin-2-yl)methyl)-2-hydroxyacetamide (ATB10052): After 2-hydroxyacetic acid (135 mg, 1.77 mmol), hydroxybenzotriazole (HOBT, 239 mg, 1.77 mmol), 1-ethyl-3-(3-dimethylaminopropyl)carbodiimide (EDCI, 340 mg, 1.77 mmol) were added to a flask containing (4,5-bis(benzyloxy)pyridine-2-yl)methanamine (500 mg, 1.47 mmol) and dissolved in dimethylformamide (DMF, 10 ml), diisopropylethylamine (DIEA, 457 mg, 3.54 mmol) was added. After the reaction solution was stirred at room temperature for 14 hours, it was added to cold water and extracted with ethyl acetate. An organic layer was washed once with brine, dehydrated with sodium sulfate (Na₂SO₄), filtered under reduced pressure, and then concentrated. The concentrated reaction solution was purified by silica gel column chromatography to synthesize N-((4,5-bis(benzyloxy)pyridin-2-yl)methyl)-2-hydroxyacetamide (ATB10052, 250 mg, 44.9% yield) in the form of a pure white solid. ¹H NMR (400 MHz, CDCl₃) δ (ppm) 4.14 (s, 2H), 4.46 (d, J=4 Hz, 2H), 5.16 (s, 2H), 5.19 (s, 2H), 6.83 (s, 1H), 7.37 (m, 11H), 8.04 (s, 1H)., ESI-MS Calcd m/z for C₂₂H₂₂N₂O₄ [M+H]⁺ 379.00 Found 378.43.

### Example 8: Preparation of 2-(((4,5-bis(benzyloxy)pyridin-2-yl)methyl)amino)ethan-1-ol (ATB10057)

After N-((4,5-bis(benzyloxy)pyridin-2-yl)methyl)-2 hydroxyacetamide (ATB10052, 250 mg, 0.661 mmol) was dissolved in tetrahydrofuran (THF, 5 ml), BH₃Me₂S (0.105 ml, 1.05 mmol, 10 mol/L) was added and stirred at 55° C. for 10 hours. After the reaction solution was cooled to room temperature, methanol (20.0 ml) was added to the reaction solution to complete the reaction, followed by concentration under reduced pressure. The concentrated solution was purified by high-resolution liquid chromatography to synthesize 2-(((4,5-bis(benzyloxy)pyridin-2-yl)methyl)amino)ethan-1-ol (ATB10057, 50 mg) in the form of a colorless oil. ¹H NMR (400 MHz, DMSO-d₆) δ (ppm) 2.63 (t, J=5.2 Hz, 2H), 3.48 (t, J=5.6 Hz, 2H), 3.78 (s, 2H), 5.17 (s, 2H), 5.22 (s, 2H), 7.23 (m, 1H), 7.31-7.48 (m, 10H), 8.13 (m, 1H), 8.23 (bs, 1H)., ESI-MS Calcd m/z for C₂₂H₂₄N₂O₃ [M]⁺ 365.5 Found 364.45.

### Preparation Example 6) the Compound of Example 9 was Synthesized by the Method Disclosed in Reaction Scheme 6 Below

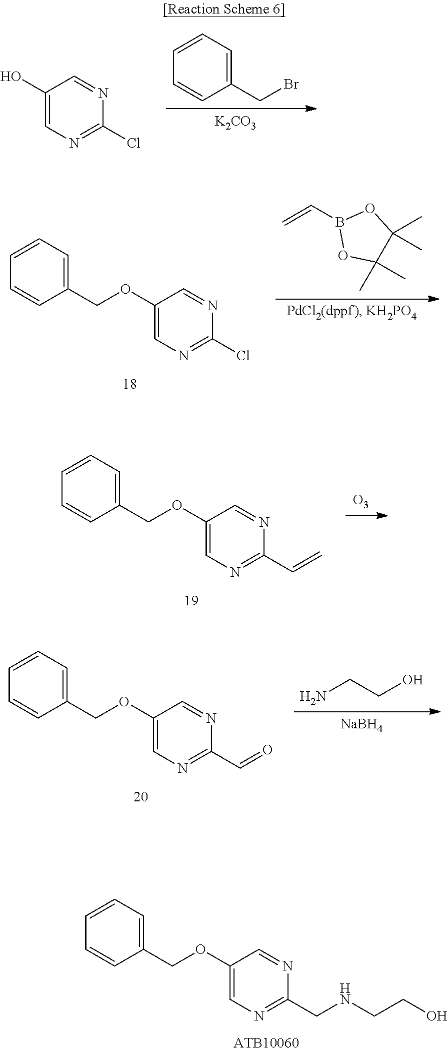

### Example 9: Preparation of 2-(((5-(benzyloxy)pyrimidin-2-yl)methyl)amino)ethan-1-ol (ATB10060)

Step 1) Synthesis of 5-(benzyloxy)-2-chloropyrimidine (18): After 2-chloropyrimidin-5-ol (4 g, 30.1 mmol) was dissolved in acetonitrile (50 ml), K₂CO₃ (8.6 g, 60.2 mmol) was added and then benzyl bromide (6.3 g, 36.7 mmol) was slowly added. The reaction solution was stirred at 60° C. for 10 hours, cooled to room temperature, and filtered under reduced pressure. The filtrate was concentrated under reduced pressure and purified by silica gel column chromatography to synthesize 5-(benzyloxy)-2-chloropyrimidine (18, 3 g) in the form of a white solid. ¹H NMR (400 MHz, DMSO_d₆) δ (ppm) 5.28 (s, 2H), 7.40-7.48 (m, 5H), 8.62 (s, 2H).

Step 2) Synthesis of 5-(benzyloxy)-2-vinylpyrimidine (19): After 5-(benzyloxy)-2-chloropyrimidine (18, 2 g, 9 mmol) was dissolved in 1,4-dioxane (30 ml) and water (5 ml), 4,4,5,5-tetramethyl-2-vinyl-1,3,2-dioxaborolane (2.1 g, 13.6 mmol), potassium phosphate (KH₂PO₄, 3.8 g, 18 mmol) and Pd(dppf)Cl₂ (0.2 g) were added to the reaction solution, and stirred at 80° C. for 14 hours. After the reaction solution was cooled to room temperature, it was filtered under reduced pressure with Celite. After the filtered solution was concentrated under reduced pressure, water and ethyl acetate were added to extract the reaction organic matter. The separated organic layer was washed once more with brine, dehydrated with sodium sulfate (Na₂SO₄), and filtered under reduced pressure, and the filtrate was concentrated to synthesize 5-(benzyloxy)-2-vinylpyrimidine (19, 0.6 g). Mass Calcd.: 212.2; MS Found: 213 [MS+1].

Step 3) Synthesis of 5-(benzyloxy)pyrimidine-2-carbaldehyde (20): After 5-(benzyloxy)-2-vinylpyrimidine (19, 0.6 g, 2.8 mmol) was dissolved in dichloromethane (50 ml), ozone was bubbled and added to the solution until the color of the reaction solution changed (blue), and then nitrogen gas was added to the reaction solution until the blue color was completely discharged. The resulting ozonide solution was cooled to −40° C., triethylamine was added, and then slowly warmed to room temperature over 1 hour. The solvent was concentrated under reduced pressure to synthesize a compound of 5-(benzyloxy)pyrimidine-2-carbaldehyde (20, 1.0 g) without further purification. Mass Calcd.: 214.2; MS Found: 214.9 [MS].

Step 4) Synthesis of 2-(((5-(benzyloxy)pyrimidin-2-yl)methyl)amino)ethan-1-ol (ATB10060): After 5-(benzyloxy)pyrimidine-2-carbaldehyde (20, 1.0 g, 4.67 mmol) was dissolved in methanol (20 ml), 2-aminoethanol (0.29 g, 4.67 mmol) was added and then stirred at 65° C. for 6 hours. After the reaction solution was cooled to room temperature, NaBH₄ (0.27 g, 7.1 mmol) was added to the reaction solution and stirred at 50° C. for 12 hours. After water was added to the reaction solution, extraction was performed three times with ethyl acetate. The obtained organic layer was washed once with brine, and then dehydrated with sodium sulfate (Na₂SO₄) and filtered under reduced pressure. The filtered solution was concentrated under reduced pressure and then purified by high-resolution liquid chromatography to synthesize 2-(((5-(benzyloxy)pyrimidin-2-yl)methyl)amino)ethan-1-ol (ATB10060, 15 mg) in the form of a pure yellow oil. ¹H NMR (400 MHz, CD₃OD) δ (ppm) 2.81 (t, J=1.6 Hz, 2H), 3.70 (t, J=5.6 Hz, 2H), 3.99 (s, 2H), 5.27 (s, 2H), 7.41 (m, 3H), 7.47 (m, 2H), 8.54 (s, 2H)., ESI-MS Calcd m/z for C₁₄H₁₇N₃O₂ [M]⁺ 259.31 Found 260.00.

### Preparation Example 7) the Compound of Example 10 was Synthesized by the Method Disclosed in Reaction Scheme 7 Below

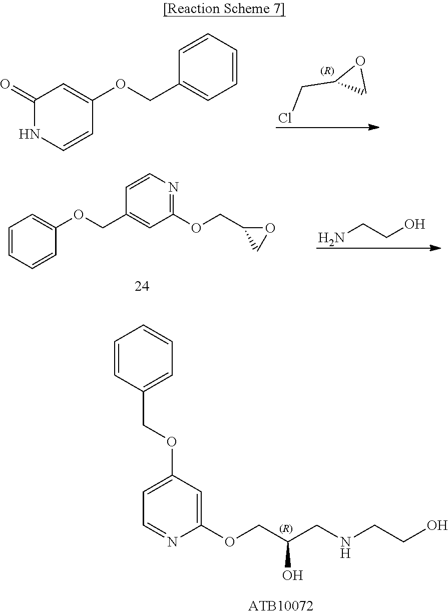

### Example 10: Preparation of (R)-1-((4-(benzyloxy)pyridin-2-yl)oxy)-3-((2-hydroxyethyl)amino)propan-2-ol (ATB10072)

Step 1) Synthesis of (R)-4-(benzyloxy)-2-(oxirane-2-ylmethoxy)pyridine (24): After 4-(benzyloxy)pyridin-2(1H)-one (1.00 g, 4.97 mmol, 1.0 eq) was dissolved in dimethylsulfoxide (DMSO, 10.0 ml), Ag₂CO₃ (1.68 g, 10.0 mmol, 2.0 eq) and NaI (0.150 g, 1.0 mmol, 0.2 eq) were added into the reaction solution while maintaining room temperature. Then, (R)-(−)-epichlorohydrin (0.93 g, 10.0 mmol, 2.0 eq) was added to the reaction solution. The reaction solution was stirred at 70° C. for 48 hours, cooled to room temperature, and then, water (50 ml) was added, and extracted three times with ethyl acetate. The obtained organic layer was washed once with brine, and then dehydrated with sodium sulfate (Na₂SO₄) and filtered under reduced pressure. The filtered solution was concentrated under reduced pressure to synthesize (R)-4-(benzyloxy)-2-(oxirane-2-ylmethoxy)pyridine (24) in the form of a yellow oil. ESI-MS Calcd m/z for C₁₅H₁₅NO₃ [M]⁺ 257.29 Found 258.1.

Step 2) Synthesis of (R)-1-((4-(benzyloxy)pyridin-2-yl)oxy)-3-((2-hydroxyethyl)amino)propan-2-ol (ATB10072): After (R)-4-(benzyloxy)-2-(oxirane-2-ylmethoxy)pyridine (24, 600 mg, 2.33 mmol, 1 eq) was dissolved in ethanol (15 ml), ethanolamine (427 mg, 7.00 mmol, 3 eq) was added and stirred at 70° C. for 16 hours. After the reaction solution was cooled to room temperature, the reaction solution was concentrated under reduced pressure and purified by high-resolution liquid chromatography to synthesize 2-(((4-(benzyloxy)pyrimidin-2-yl)methyl)amino)ethan-1-ol (ATB10061, 15 mg) in the form of a pure white solid. ¹H NMR (CD₃OD, 500 MHz): δ (ppm) 7.35-7.53 (m, 6H), 6.18 (m, 1H), 6.04 (m, 1H), 5.12 (s, 2H), 4.17-4.19 (m, 1H), 4.05 (m, 1H), 3.75-3.78 (m, 1H), 3.66-3.69 (m, 2H), 2.62-2.78 (m, 4H)., ESI-MS Calcd m/z for C₁₇H₂₂N₂O₄ [M]⁺ 318.37 Found 319.0.

### Preparation Example 8) the Compound of Example 11 was Synthesized by the Method Disclosed in Reaction Scheme 8 Below

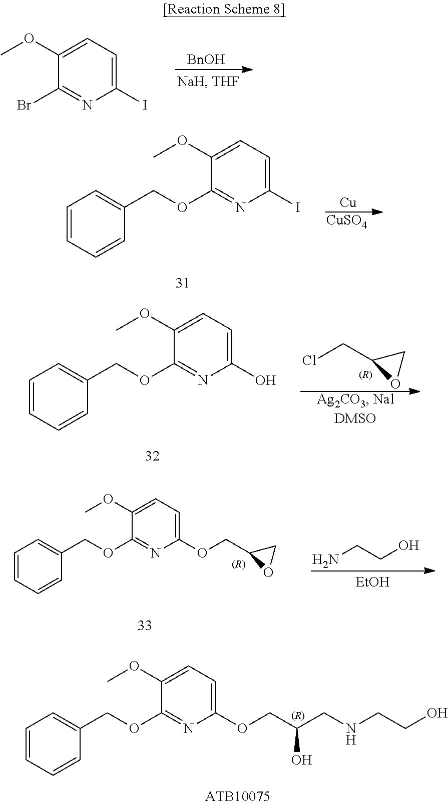

### Example 11: Preparation of (R)-1-((6-(benzyloxy)-5-methoxypyridin-2-yl)oxy)-3-((2-hydroxyethyl)amino)propan-2-ol (ATB10075)

Step 1) Synthesis of 2-(benzyloxy)-6-iodo-3-methoxypyridine (31): After NaH (0.96 g, 24.0 mmol, 1.5 eq) was dissolved in anhydrous tetrahydrofuran (THF, 50 ml) under anhydrous conditions, benzyl alcohol (2.60 gg, 24.0 mmol, 1.5 eq) was slowly added dropwise to the reaction solution at 20° C. The reaction mixture was stirred for 30 minutes, and cooled to 0° C., and the solution of 2-bromo-6-iodo-3-methoxypyridine (5.0 g, 16.0 mmol, 1.0 eq) dissolved in anhydrous tetrahydrofuran (THF, 10 ml) was added slowly. The reaction mixture was stirred at 60° C. for 5 hours. When the reaction was complete, the reaction solution was added to ice water and extracted three times with ethyl acetate (50 ml). The obtained organic layer was washed with brine, dehydrated with Na₂SO₄ and filtered under reduced pressure. The filtered solution was concentrated to synthesize 2-(benzyloxy)-6-iodo-3-methoxypyridine (31, 2.80 g) in the form of a yellow solid. ESI-MS Calcd m/z for C₁₃H₁₂INO₂ [M]⁺ 341.1 Found 341.9 and 342.9.

Step 2) Synthesis of 6-(benzyloxy)-5-methoxypyridin-2-ol (32):

2-(Benzyloxy)-6-iodo-3-methoxypyridine (31, 2.8 g, 8.21 mmol, 1.0 eq), NaOH (1.65 g, 41.1 mmol, 5.0 eq), Cu (0.56 g, 10 mmol, 1.22 eq) and CuSO₄.5H₂O (0.56 g, 2.24 mmol, 0.27 eq) were dissolved in DMSO (40 mL) and H₂O (2 mL), and then stirred at 90° C. for 16 hours. Upon completion of the reaction, the reaction solution was concentrated under reduced pressure and purified by column chromatography (PE/EA=5/1 to 3/1) to synthesize 6-(benzyloxy)-5-methoxypyridin-2-ol (32, 1.5 g) in the form of a white solid. ESI-MS Calcd m/z for C13H13NO3 [M]⁺ 231.2 Found 232.1.

Step 3) Synthesis of (R)-2-(benzyloxy)-3-methoxy-6-(oxirane-2-ylmethoxy)pyridine (33): After 6-(benzyloxy)-5-methoxypyridine-2-ol (32, 270 mg, 1.16 mmol, 1.0 eq), (R)-(−)-epichlorohydrin (215 mg, 2.32 mmol, 2.0 eq), Ag₂CO₃ (640 mg, 2.32 mmol, 2.0 eq) and NaI (17.4 mg, 0.116 mmol, 0.1 eq) were dissolved in dimethylformamide (DMF, 10 mL), the solution was stirred at 70° C. for 48 hours. When the reaction was complete, the reaction solution was diluted with water (50 ml) and extracted three times with ethyl acetate (50 ml). The obtained organic layer was washed with brine, dehydrated with sodium sulfate (Na₂SO₄) and filtered under reduced pressure. The filtered solution was concentrated to synthesize (R)-2-(benzyloxy)-3-methoxy-6-(oxirane-2-ylmethoxy)pyridine (33, 270 mg) in the form of a yellow oil. ESI-MS Calcd m/z for C₁₆H₁₇NO₄ [M]⁺ 287.3 Found 288.1 and 289.1.

Step 4) synthesis of (R)-1-((6-(benzyloxy)-5-methoxypyridin-2-yl)oxy)-3-((2-hydroxyethyl)amino)propan-2-ol (ATB10075)

After (R)-2-(benzyloxy)-3-methoxy-6-(oxirane-2-ylmethoxy)pyridine (33, 270 mg, 0.94 mmol, 1 eq) and ethanolamine (172 mg, 2.82 mmol, 3 eq) were dissolved in ethanol (10 mL), it was stirred at 70° C. for 16 hours. After the reaction solution was cooled to room temperature, it was concentrated under reduced pressure and purified by high-resolution liquid chromatography to synthesize (R)-1-((6-(benzyloxy)-5-methoxypyridin-2-yl)oxy)-3-((2-hydroxyethyl)amino)propan-2-ol (ATB10075, 15 mg, 4.5%) in the form of a pure white solid.

¹HNMR (CD₃OD, 400 MHz): δ 7.45-7.47 (m, 2H), 7.28-7.39 (m, 4H), 6.32 (d, J=8.4 Hz, 1H), 5.43 (s, 2H), 4.16-4.24 (m, 2H), 4.09-4.12 (m, 1H), 3.80 (s, 3H), 3.69-3.73 (m, 2H), 2.75-2.91 (m, 4H)., ESI-MS Calcd m/z for C18H24N2O5 [M+H]⁺ 348.4 Found 349.

### Preparation Example 9) the Compound of Example 12 was Synthesized by the Method Disclosed in Reaction Scheme 9 Below

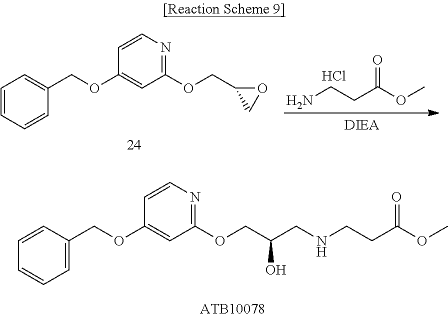

### Example 12: Preparation of methyl (R)-3-((3-((4-(benzyloxy)pyridin-2-yl)oxy)-2-hydroxypropyl)amino)propanoate (ATB10078)

After (R)-4-(benzyloxy)-2-(oxirane-2-ylmethoxy)pyridine (24, 500 mg, 1.94 mmol, 1 eq) synthesized according to the synthesis method disclosed in Reaction Scheme 9 was dissolved in methanol (15 ml), methyl 3-aminopropanoate hydrochloride (540 mg, 3.88 mmol, 2 eq) and diisopropylethylamine (DIEA, 500 mg, 3.88 mmol, 2 eq) were sequentially added thereto and stirred at 60° C. for 16 hours. After the reaction solution was cooled to room temperature, it was concentrated under reduced pressure and purified by high-resolution liquid chromatography to synthesize methyl (R)-3-((3-((4-(benzyloxy)pyridin-2-yl)oxy)-2-hydroxypropyl)amino)propanoate (ATB10078, 21 mg) in the form of a pure white solid. ¹HNMR (CDCl₃, 400 MHz): δ (ppm) 7.27-7.40 (m, 6H), 5.98-6.01 (m, 2H), 4.99 (s, 2H), 4.15-4.19 (m, 1H), 3.92-3.96 (m, 1H), 3.82-3.87 (m, 1H), 3.69 (s, 3H), 2.89-2.93 (m, 2H), 2.74-2.78 (m, 1H), 2.50-2.61 (m, 3H)., ESI-MS Calcd m/z for C₁₉H₂₄N₂O₅ [M]⁺ 360.4 Found 361.

### Preparation Example 10) the Compound of Example 13 was Synthesized by the Method Disclosed in Reaction Scheme 10 Below

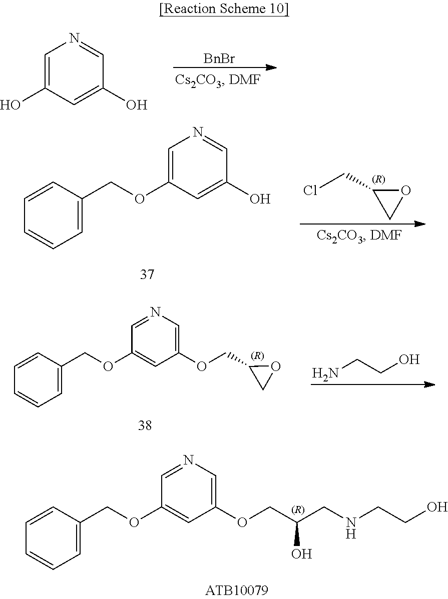

### Example 13: Preparation of (R)-1-((5-(benzyloxy)pyridin-3-yl)oxy)-3-((2-hydroxyethyl)amino)propan-2-ol (ATB10079)

Step 1) Synthesis of 5-(benzyloxy)pyridin-3-ol (37): After pyridine-3,5-diol (1.0 g, 9.01 mmol, 1.0 eq), benzyl bromide (770 mg, 4.5 mmol, 0.5 eq) and cesium carbonate (Cs₂CO₃, 4.4 g, 13.5 mmol, 1.5 eq) were dissolved in dimethylformamide (DMF, 40 ml), it was stirred at 10° C. for 16 hours, and then the precipitated solid was filtered. The filtered solid was washed with a small amount of dimethylformamide (DMF) and dried under reduced pressure to obtain 5-(benzyloxy)pyridin-3-ol (37, 1.2 g) in the form of a yellow solid. ESI-MS Calcd m/z for C₁₂H₁₁NO₂ [M]⁺ 201.2 Found 202.

Step 2) Synthesis of (R)-3-(benzyloxy)-5-(oxirane-2-ylmethoxy)pyridine (38): After 5-(benzyloxy)pyridin-3-ol (37, 1.1 g, 5.47 mmol, 1.0 eq) and cesium carbonate (Cs₂CO₃, 5.34 g, 16.4 mmol, 3.0 eq) were dissolved in dimethylformamide (DMF, 15 ml), (R)-2-(chloromethoxy)oxirane (1.52 g, 16.4 mmol, 3.0 eq) was added to the solution and stirred at 40° C. for 16 hours. After the reaction solution was cooled to room temperature, when the reaction was not completed, (R)-2-(chloromethoxy)oxirane (1.52 g, 16.4 mmol, 3.0 eq) was added and further stirred at 40° C. for 16 hours. After the reaction solution was cooled to room temperature, the reaction solution was filtered under reduced pressure, and the resulting filtrate was concentrated under reduced pressure, and (R)-3-(benzyloxy)-5-(oxirane-2-ylmethoxy)pyridine (38, 1.0 g) in the form of a yellow solid was obtained. ESI-MS Calcd m/z for C₁₅H₁₅NO₃ [M]⁺ 257.2 Found 258.

Step 3) Synthesis of (R)-1-((5-(benzyloxy)pyridin-3-yl)oxy)-3-((2-hydroxyethyl)amino)propan-2-ol (ATB10079): After (R)-3-(benzyloxy)-5-(oxirane-2-ylmethoxy)pyridine (38, 1.0 g, 3.89 mmol, 1.0 eq) and ethanolamine (0.71 g, 11.7 mmol, 3.0 eq) were dissolved in ethanol (10 ml), it was stirred at 40° C. for 6 hours. After the reaction solution was cooled to room temperature, it was concentrated under reduced pressure and purified by high-resolution liquid chromatography to synthesize (R)-1-((5-(benzyloxy)pyridin-3-yl)oxy)-3-((2-hydroxyethyl)amino)propan-2-ol (ATB10079, 22 mg) in the form of a pure yellow oil. ¹HNMR (CD₃OD, 400 MHz): δ (ppm) 8.49 (s, 1H), 7.67 (s, 1H), 7.55 (s, 1H), 7.45-7.47 (m, 5H), 6.96 (s, 1H), 5.48 (s, 2H), 4.27-4.30 (m, 1H), 4.08-4.10 (m, 2H), 3.84 (t, J=4.8 Hz, 2H), 3.29-3.30 (d, J=2.8 Hz, 1H), 3.14-3.21 (m, 3H)., ESI-MS Calcd m/z for C₁₇H₂₂N₂O₄ [M]⁺ 318.3 Found 319.

### Preparation Example 11) the Compound of Example 14 was Synthesized by the Method Disclosed in Reaction Scheme 11 Below

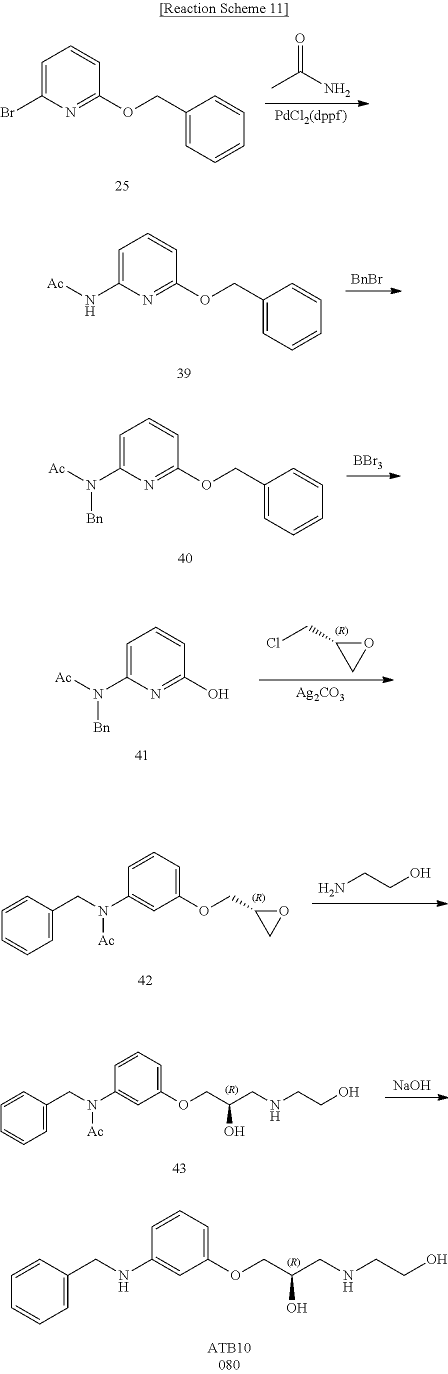

### Example 14: Preparation of (R)-1-((6-(benzylamino)pyridin-2-yl)oxy)-3-((2-hydroxyethyl)amino)propan-2-ol (ATB10080)

Step 1) Synthesis of N-(6-(benzyloxy)pyridin-2-yl)acetamide (39): After 2-(benzyloxy)-6-bromopyridine (25, 5.0 g, 19.0 mmol, 1 eq), acetamide (1.2 g, 20.0 mmol, 1.1 eq), PdCl₂ (dppf) (139 mg, 1.9 mmol, 0.1 eq) and X-Phos (100 mg, 2 mmol, 1.1 eq) were dissolved in toluene (50 ml), it was stirred under reflux for 17 hours. After the reaction solution was cooled to room temperature, it was diluted with water (80 ml) and extracted three times with ethyl acetate (50 ml). The obtained organic layer was washed with brine, dehydrated with sodium sulfate (Na₂SO₄), filtered under reduced pressure, and the filtered solution was concentrated to obtain N-(6-(benzyloxy)pyridin-2-yl)acetamide (39, 3 g) in the form of a yellow solid. ¹H NMR (CDCl₃, 400 MHz): δ (ppm) 7.74-7.75 (m, 1H), 7.43-7.65 (m, 2H), 7.30-7.43 (m, 5H), 6.55 (d, J=8.0 Hz, 1H), 5.28 (s, 2H), 2.20 (s, 3H)., ESI-MS Calcd m/z for C₁₄H₁₄N₂O₂ [M]⁺ 242.2 Found 243.

Step 2) Synthesis of N-benzyl-N-(6-(benzyloxy)pyridin-2-yl)acetamide (40): After N-(6-(benzyloxy)pyridin-2-yl)acetamide (39, 7 g, 28.9 mmol, 1 eq), benzyl bromide (5.3 g, 28.9 mmol, 1 eq) and cesium carbonate (Cs₂CO₃, 18.8 g, 57.8 mmol, 2 eq) were dissolved in dimethylformamide (DMF, 140 ml), it was stirred at 25° C. for 10 hours. The reaction solution was diluted in water (600 ml) and extracted three times with ethyl acetate (150 ml). The obtained organic layer was washed with brine, dehydrated with sodium sulfate (Na₂SO₄), filtered under reduced pressure, and the filtered solution was concentrated to synthesize N-benzyl-N-(6-(benzyloxy)pyridin-2-yl)acetamide (40, 7 g, crude) in the form of a yellow solid. ESI-MS Calcd m/z for C₂₁H₂ON₂O₂ [M]⁺ 332.4 Found 333.

Step 3) Synthesis of N-benzyl-N-(6-hydroxypyridin-2-yl)acetamide (41): After N-benzyl-N-(6-(benzyloxy)pyridin-2-yl)acetamide (40, 7.0 g, 21.6 mmol, 1 eq) was dissolved in dichloromethane (DCM, 200 ml), it was cooled to −20° C. Then, boron tribromide (BBr₃, 8.1 g, 32.7 mmol, 1.5 eq) was slowly added dropwise to the reaction solution, and then, it was stirred at room temperature for 4 hours. After the reaction solution was put in cold water (200 ml), it was extracted three times with dichloromethane (DCM, 50 ml). The obtained organic layer was washed with brine, dehydrated with sodium sulfate (Na₂SO₄), filtered under reduced pressure, and the filtered solution was concentrated to synthesize N-benzyl-N-(6-hydroxypyridin-2-yl) acetamide (41, 2.5 g) in the form of a gray solid. ESI-MS Calcd m/z for C₁₄H₁₄N₂O₂ [M]⁺ 242.28 Found 243.

Step 4) Synthesis of (R)—N-benzyl-N-(6-(oxirane-2-ylmethoxy)pyridin-2-yl)acetamide (42): After N-benzyl-N-(6-hydroxylpyridine-2-yl)acetamide (41, 2.2 g, 9.1 mmol, 1 eq), (R)-2-(chloromethoxy) oxirane (827 mg, 91 mmol, 10 eq), silver carbonate (Ag₂CO₃, 5 g, 18.2 mmol, 2 eq) and NaI (3.3 g, 18.2 mmol, 2 eq) were dissolved in dimethyl sulfoxide (DMSO, 22 ml), it was stirred at 55° C. for 24 hours. When the reaction was complete, the reaction solution was cooled to room temperature, diluted with water (200 ml), and extracted three times with ethyl acetate (50 ml). The obtained organic layer was washed with brine, dehydrated with sodium sulfate (Na₂SO₄), filtered under reduced pressure, and the filtered solution was concentrated to synthesize (R)—N-benzyl-N-(6-(oxirane-2)-ylmethoxy)pyridin-2-yl) acetamide (42, 2.7 g) in the form of a yellow oil. ESI-MS Calcd m/z for C₁₇H₁₈N₂O₃ [M]⁺ 298.3 Found 299.

Step 5) Synthesis of (R)—N-benzyl-N-(6-(2-hydroxy-3-((2-hydroxyethyl)amino)propoxy)pyridin-2-yl)acetamide (43): After (R)—N-benzyl-N-(6-(oxirane-2-ylmethoxy)pyridin-2-yl)acetamide (42, 2.7 g, 9.1 mmol, 1 eq) and ethanolamine (1.7 g, 27.2 mmol, 3 eq) were dissolved in ethanol (30 ml), it was stirred at 55° C. for 4 hours. After the reaction solution was cooled to room temperature, it was concentrated under reduced pressure and purified by high-resolution liquid chromatography to synthesize (R)—N-benzyl-N-(6-(2-hydroxy-3-((2-hydroxyethyl))amino) propoxy) pyridin-2-yl) acetamide (43, 3.0 g) in the form of a pure yellow oil. ¹HNMR (CDCl₃, 400 MHz): δ (ppm) 7.54-7.59 (m, 1H), 7.22-7.29 (m, 6H), 6.64-6.72 (m, 2H), 5.22 (br s, 1H), 5.03 (s, 2H), 4.11-4.40 (m, 5H), 3.73 (s, 2H), 3.36-3.37 (m, 2H), 3.08 (br s, 1H), 2.11 (s, 3H)., ESI-MS Calcd m/z for C₁₉H₂₅N₃O₄ [M]⁺ 359.4 Found 360.

Step 6) Synthesis of (R)-1-((6-(benzylamino)pyridin-2-yl)oxy)-3-((2-hydroxyethyl)amino)propan-2-ol (ATB10080): After (R)—N-benzyl-N-(6-(2-hydroxy-3-((2-hydroxyethyl)amino)propoxy)pyridin-2-yl)acetamide (43, 3.0 g, 8.36 mmol, 1 eq) was dissolved in methanol (45 ml), NaOH (668 mg, 16.7 mmol, 2 eq) was added and stirred at 55° C. for 4 hours. When the reaction was complete, the reaction solution was cooled to room temperature, diluted with water (150 ml), and extracted three times with ethyl acetate (50 ml). The obtained organic layer was washed with brine, dehydrated with sodium sulfate (Na₂SO₄), filtered under reduced pressure, and the filtered solution was concentrated and purified by high-resolution liquid chromatography to synthesize (R)-1-((6)-(benzylamino)pyridin-2-yl)oxy)-3-((2-hydroxyethyl)amino)propan-2-ol (ATB10080, 330 mg) in the form of a pure yellow oil. ¹H NMR (CD₃OD, 400 MHz): δ (ppm) 7.35-7.37 (m, 2H), 7.28-7.32 (m, 3H), 7.21 (t, J=7.2 Hz, 1H), 6.04 (d, J=8.0 Hz, 1H), 5.97 (d, J=8.0 Hz, 1H), 4.50 (s, 2H), 4.16-4.20 (m, 2H), 4.02-4.08 (m, 1H), 3.64-3.72 (m, 2H), 2.66-2.81 (m, 4H)., ESI-MS Calcd m/z for C₁₉H₂₅N₃O₄ [M]⁺ 317 Found 318.

### Preparation Example 12) the Compound of Example 15 was Synthesized by the Method Disclosed in Reaction Scheme 12 Below

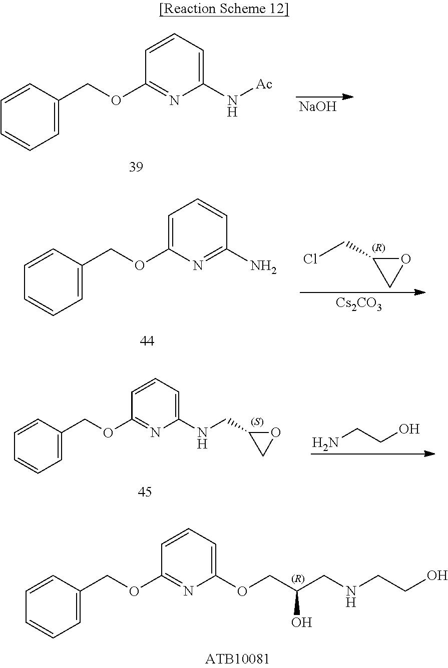

### Example 15: Preparation of (R)-1-((6-(benzyloxy)pyridin-2-yl)amino)-3-((2-hydroxyethyl)amino)propan-2-ol (ATB10081)

Step 1) Synthesis of 6-(benzyloxy)pyridin-2-amine (44): After N-(6-(benzyloxy)pyridin-2-yl)acetamide (39, 6 g, 24.8 mmol, 1 eq) and NaOH (1 g, 24.8 mmol, 1 eq) were dissolved in methanol (90 ml), it was stirred at 60° C. for 10 hours. When the reaction was complete, the reaction solution was cooled to room temperature, diluted with water (300 ml), and extracted three times with ethyl acetate (100 ml). The obtained organic layer was washed with brine, dehydrated with sodium sulfate (Na₂SO₄), filtered under reduced pressure, and the filtered solution was concentrated to synthesize 6-(benzyloxy)pyridin-2-amine (44, 4.7 g) in the form of a yellow oil. ESI-MS Calcd m/z for C₁₂H₁₂N₂O [M]⁺ 200.2 Found 201.

Step 2) Synthesis of (R)-6-(benzyloxy)-N-(oxirane-2-ylmethyl)pyridin-2-amine (45): After 6-(benzyloxy)pyridin-2-amine (44, 4.7 g, 23.5 mmol, 1 eq), (R)-2-(chloromethyl) oxirane (2.2 g, 235 mmol, 10 eq) and Cs₂CO₃ (23.0 g, 70.5 mmol, 3 eq) were dissolved in dimethylformamide (DMF, 100 ml), it was stirred at 65° C. for 10 hours. When the reaction was complete, the reaction solution was cooled to room temperature, diluted with water (300 ml), and extracted three times with ethyl acetate (100 ml). The obtained organic layer was washed with brine, dehydrated with sodium sulfate (Na₂SO₄), filtered under reduced pressure, and the filtered solution was concentrated to synthesize (R)-6-(benzyloxy)-N-(oxirane-2-ylmethyl)pyridin-2-amine (45, 6.6 g) in the form of a yellow. ESI-MS Calcd m/z for C₁₅H₁₆N₂O₂ [M]⁺ 256.3 Found 257.

Step 3) Synthesis of (R)-1-((6-(benzyloxy)pyridin-2-yl)amino)-3-((2-hydroxyethyl)amino)propan-2-ol (ATB10081): After (R)-6-(benzyloxy)-N-(oxirane-2-ylmethyl)pyridin-2-amine (45, 6.6 g, 25.8 mmol, 1 eq) was dissolved in ethanol (100 ml), ethanolamine (4.7 g, 77.4 mmol, 3 eq) was added and stirred at 60° C. for 4 hours. When the reaction was complete, the reaction solution was cooled to room temperature, and the reaction solution was concentrated and purified by high-resolution liquid chromatography to synthesize (R)-1-((6-(benzyloxy)pyridin-2-yl)amino)-3-((2-hydroxyethyl)amino)propan-2-ol (ATB10081, 650 mg) in the form of a pure yellow oil. ¹H NMR (CD₃OD, 400 MHz): δ (ppm) 8.55 (s, 1H), 7.43-7.45 (m, 2H), 7.38 (t, J=6.4 Hz, 3H), 7.31-7.33 (m, 1H), 6.13 (d, J=8.0 Hz, 1H), 6.07 (d, J=8.0 Hz, 1H), 5.28 (s, 2H), 4.07-4.10 (m, 1H), 3.76 (t, J=4.8 Hz, 2H), 3.446-3.453 (m, 2H), 3.07-3.16 (m, 3H), 2.94-2.99 (m, 1H)., ESI-MS Calcd m/z for C₁₇H₂₃N₃O₃ [M]⁺ 317.3 Found 318.

### Preparation Example 13) the Compound of Example 16 was Synthesized by the Method Disclosed in Reaction Scheme 13 Below

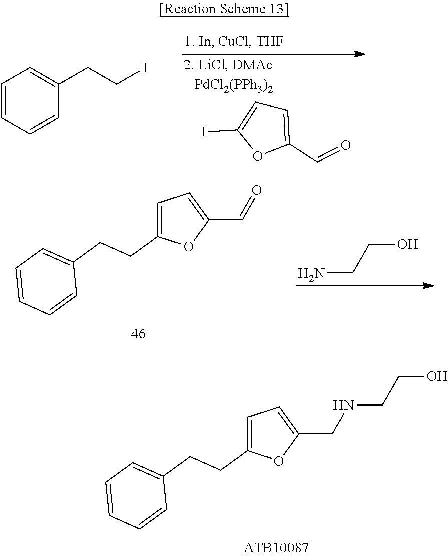

### Example 16: Preparation of 2-(((5-phenethylfuran-2-yl)methyl)amino)ethan-1-ol (ATB10087)

Step 1) Synthesis of 5-phenethylfuran-2-carbaldehyde (46): After (2-iodoethyl)benzene (1.00 g, 4.3 mmol, 1.0 eq), In (0.99 g, 8.6 mmol, 2.0 eq) and CuCl (0.85 g, 8.6 mmol, 2.0 eq) were dissolved in tetrahydrofuran (THF, 20 ml), it was stirred at 25° C. for 24 hours. When the reaction was complete, the stirring was stopped for about 10 minutes so that a precipitate was formed. The transparent solution at the top of the reaction solution was separated from the black precipitate, and the black precipitate was washed once with tetrahydrofuran (THF, 10 ml) to separate the residual THF solution again. After the separated THF solution layer was concentrated under reduced pressure, N,N-dimethylacetamide (DMAc, 20 ml) was added and dissolved, and 5-iodofuran-2-carbaldehyde (0.96 g, 4.3 mmol, 1.0 eq), LiCl (0.37 g, 8.6 mmol, 2.0 eq) and PdCl₂(PPh₃)₂ (0.15 g, 0.22 mmol, 0.05 eq) were sequentially added. The reaction solution was stirred at 100° C. for 24 hours. When the reaction was complete, the reaction solution was cooled to room temperature, diluted with water, and extracted with ethyl acetate (EA). The extracted organic layer was washed once more with brine, dehydrated with sodium sulfate (Na₂SO₄), filtered under reduced pressure, and concentrated. The concentrated mixture was purified by column chromatography to synthesize 5-phenethylfuran-2-carbaldehyde (46, 0.31 g) in the form of a yellow oil. ¹H NMR (CDCl₃, 400 MHz): δ (ppm) 9.53 (s, 1H), 7.26-7.30 (m, 2H), 7.14-7.22 (m, 4H), 6.18 (d, J=3.6 Hz, 1H), 2.99-3.07 (m, 4H).

Step 2) Synthesis of 2-(((5-phenethylfuran-2-yl)methyl)amino)ethan-1-ol (ATB10087): After 5-phenethylfuran-2-carbaldehyde (46, 50 mg, 0.25 mmol, 1.0 eq) and 2-aminoethanol (30 mg, 0.5 mmol, 2 eq) were dissolved in methanol (5 ml), 1 drop of acetic acid was added, and stirred at room temperature for 1 hour. When imine was formed, NaBH₄ (19 mg, 0.5 mmol, 2 eq) was added to the reaction solution and stirred at room temperature for an additional hour. When the reaction was completed, the reaction solution was diluted in water (50 ml), extracted three times with ethyl acetate (EA, 50 ml), and then an organic layer was washed once more with brine, and then dehydrated with sodium sulfate (Na₂SO₄) and concentrated by filtration under reduced pressure. The concentrated solution was purified by high-resolution liquid chromatography to system 2-(((5-phenethylfuran-2-yl)methyl)amino)ethan-1-ol (ATB10087, 28 mg, 45.7% yield) in the form of a pure white solid. ¹H NMR (CDCl₃, 400 MHz): δ (ppm) 7.26-7.30 (m, 2H), 7.17-7.21 (m, 3H), 6.05 (d, J=3.2 Hz, 1H), 5.89 (d, J=3.2 Hz, 1H), 3.75 (s, 2H), 3.65 (t, J=3.2 Hz, 2H), 2.89-2.96 (m, 4H), 2.78 (t, J=3.2 Hz, 2H), 1.99 (brs, 2H)., ESI-MS Calcd m/z for C₁₅H₁₉NO₂ [M]⁺ 245.3 Found 246.

### Preparation Example 14) the Compound of Example 17 was Synthesized by the Method Disclosed in Reaction Scheme 14 Below

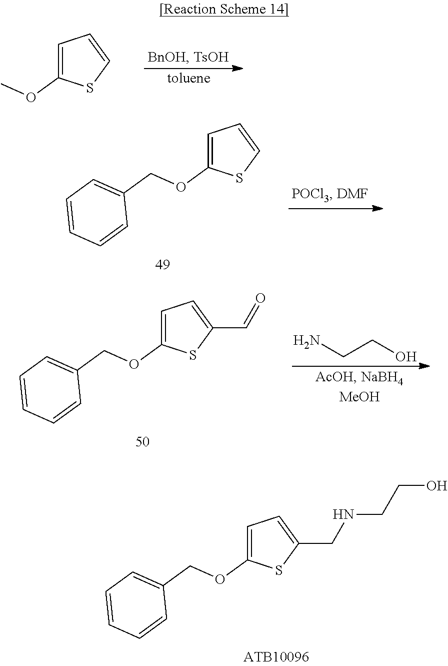

### Example 17: Preparation of 2-(((5-(benzyloxy)thiophen-2-yl)methyl)amino)ethan-1-ol (ATB10096)

Step 1) Synthesis of 2-(benzyloxy)thiophene (49): After 2-methoxythiophene (3.0 g, 26 mmol, 1.0 eq) was dissolved in toluene (60 ml), it was cooled to 10° C., and benzyl alcohol (7.1 g, 66 mmol, 2.5 eq) and p-toluenesulfonic acid (0.45 g, 2.6 mmol, 0.1 eq) were sequentially added to the reaction solution, and stirred at 90° C. for 1 hour. After water was added to the reaction solution, it was extracted with ethyl acetate (EA). The extracted organic layer was washed once more with brine, dehydrated with sodium sulfate (Na₂SO₄) and concentrated by filtration under reduced pressure, and then purified by column chromatography to synthesize 2-(benzyloxy)thiophene (49, 1.4 g) in the form of a colorless oil. ¹H NMR (CDCl₃, 400 MHz): δ (ppm) 7.32-7.43 (m, 5H), 6.71 (dd, J=6.4, 3.6 Hz, 1H), 6.56 (dd, J=5.2, 1.2 Hz, 1H), 6.27 (dd, J=3.6, 1.2 Hz, 1H), 5.07 (s, 2H).

Step 2) Synthesis of 5-(benzyloxy)thiophene-2-carbaldehyde (50): After the reaction vessel containing dimethylformamide (DMF, 20 ml) was cooled to 10° C., POCl3 (1.61 g, 10.5 mmol, 5.0 eq) was slowly added to the reaction vessel and stirred for 1 hour. After 2-(benzyloxy)thiophene (49, 0.40 g, 2.10 mmol, 1.0 eq) was dissolved in 2 ml of dimethylformamide (DMF), it was slowly added to the reaction solution while maintaining 10° C. After the reaction mixture was stirred for 30 minutes, 10N NaOH aqueous solution was added until the pH value reached 9 while maintaining the temperature of 0 to 5° C. After the reaction solution was stirred for an additional 1 hour, it was diluted with water (60 ml) and extracted three times with ethyl acetate (EA, 30 ml). The extracted organic layer was washed once more with brine, dehydrated with sodium sulfate (Na₂SO₄) and concentrated by filtration under reduced pressure, and then purified by column chromatography to synthesize 5-(benzyloxy)thiophene-2-carbaldehyde (50, 0.40 g) in the form of a pure yellow solid. ¹H NMR (CDCl₃, 400 MHz): δ (ppm) 9.67 (s, 1H), 7.51 (d, J=4.0 Hz, 1H), 7.40-7.50 (m, 5H), 6.41 (d, J=4.4 Hz, 1H), 5.18 (s, 2H).

Step 3) Synthesis of 2-(((5-(benzyloxy)thiophen-2-yl)methyl)amino)ethan-1-ol (ATB10096): After 5-(benzyloxy)thiophene-2-carbaldehyde (50, 150 mg, 0.69 mmol, 1.0 eq) was dissolved in methanol (10 ml), ethanolamine (84 mg, 1.38 mmol, 2.0 eq) and acetic acid (3 drops) were sequentially added, and stirred at 30° C. for 1 hour. When imine is formed, NaBH₄ (52 mg, 1.38 mmol, 2.0 eq) was slowly added to the reaction solution, and further stirred for 1 hour, and water was added to complete the reaction. The reaction solution was concentrated under reduced pressure, diluted with ethyl acetate (EA), washed with water, and then the organic layer was dehydrated with sodium sulfate (Na₂SO₄) and filtered under reduced pressure to concentrate. The concentrated solution was purified with prep-TLC (DCM/MeOH=5/1) to obtain 2-(((5-(benzyloxy)thiophen-2-yl)methyl)amino)ethane-1-ol (ATB10096, 50 mg) in the form of a pure yellow oil. ¹H NMR (CDCl₃, 400 MHz): δ (ppm) 7.34-7.43 (m, 5H), 6.55 (d, J=3.6 Hz, 1H), 6.09 (d, J=3.6 Hz, 1H), 5.05 (s, 2H), 3.88 (s, 2H), 3.66 (t, J=4.8 Hz, 2H), 2.83 (t, J=5.2 Hz, 2H), 2.39 (br s, 2H)., ESI-MS Calcd m/z for C₁₄H₁₇NO₂S [M]⁺ 263.3 Found 203 [MS-60].

### Preparation Example 15) the Compound of Example 18 was Synthesized by the Method Disclosed in Reaction Scheme 15 Below

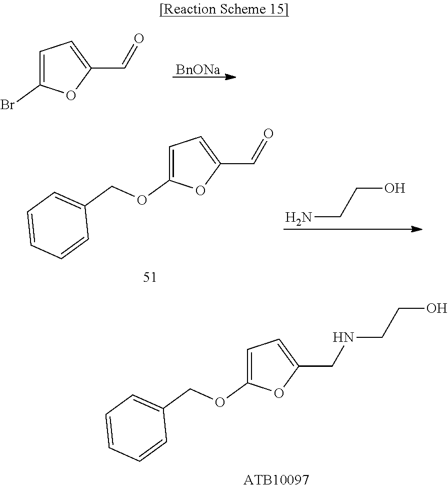

### Example 18: Preparation of 2-(((5-(benzyloxy)-furan-2-yl)methyl)amino)ethan-1-ol (ATB10097)

Step 1) Synthesis of 5-(benzyloxy)furan-2-carbaldehyde (51): After 5-bromofuran-2-carbaldehyde (2.00 g, 11.5 mmol, 1.0 eq) was diluted to benzyl alcohol (20 ml), it was cooled to 10° C. Then, after BnONa (2.00 g, 5.7 mmol, 3 mol/L in BnOH, 0.5 eq) was added to the solution, it was heated to 80° C., and stirred under N₂ conditions for 12 hours. Water was added to the reaction solution, and the reaction solution was extracted with ethyl acetate (EA). The extracted organic layer was washed once more with brine, dehydrated with sodium sulfate (Na₂SO₄) and concentrated by filtration under reduced pressure, and then purified by column chromatography to synthesize 5-(benzyloxy)furan-2-Carbaldehyde (51, 0.30 g) in the form of a pure brown solid. ¹H NMR (CDCl₃, 400 MHz): δ (ppm) 9.31 (s, 1H), 7.37-7.41 (m, 5H), 7.20 (d, J=3.6 Hz, 1H), 5.51 (d, J=3.6 Hz, 1H), 5.28 (s, 2H).

Step 2) Synthesis of 2-(((5-(benzyloxy)-furan-2-yl)methyl)amino)ethan-1-ol (ATB10097): After 5-(benzyloxy)furan-2-carbaldehyde (51, 200 mg, 0.99 mmol, 1.0 eq) was dissolved in methanol (5 ml), ethanolamine (120 mg, 1.98 mmol, 2.0 eq) and acetic acid (1 drop) were sequentially added and stirred at 30° C. for 1 hour. When imine was formed, NaBH₄ (75 mg, 1.98 mmol, 2.0 eq) was slowly added to the reaction solution, and then further stirred for 1 hour. After that, water was added to complete the reaction. The reaction solution was concentrated under reduced pressure, diluted with ethyl acetate (EA), washed with water, and then the organic layer was dehydrated with sodium sulfate (Na₂SO₄) and filtered under reduced pressure to concentrate. The concentrated solution was purified with prep-TLC (DCM/MeOH=10/1) and 2-(((5-(benzyloxy)-furan-2-yl)methyl)amino)ethane-1-ol (ATB10097, 46 mg) in the form of a pure brown oil was obtained. ¹H NMR (CDCl₃, 400 MHz): δ (ppm) 7.34-7.41 (m, 5H), 6.04 (d, J=3.2 Hz, 1H), 5.11 (d, J=3.2 Hz, 1H), 5.05 (s, 2H), 3.69 (s, 2H), 3.63 (t, J=5.2 Hz, 2H), 2.77 (t, J=5.2 Hz, 2H)., ESI-MS Calcd m/z for C₁₄H₁₇NO₃ [M]⁺ 247.3 Found 91.1, 186.9 [MS-156, MS-60].

### Preparation Example 16) the Compound of Example 19 was Synthesized by the Method Disclosed in Reaction Scheme 16 Below

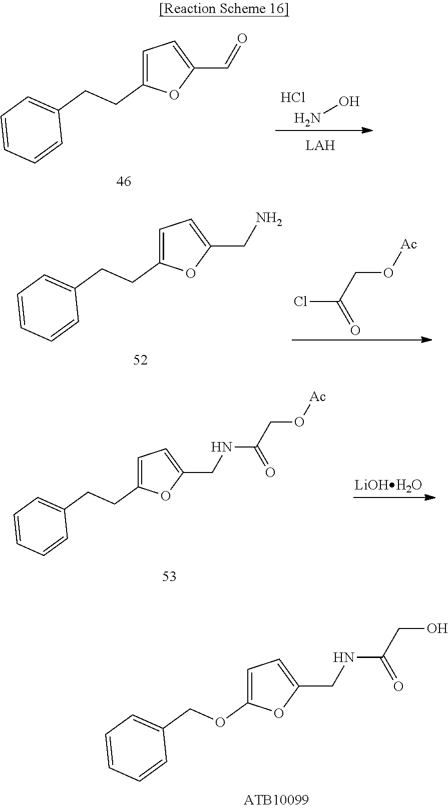

### Example 19: Preparation of 2-hydroxy-N-((5-phenethylfuran-2-yl)methyl) acetamide (ATB10099)

Step 1) Synthesis of (5-phenethylfuran-2-yl)methanamine (52): After 5-phenethylfuran-2-carbaldehyde (46, 400 mg, 1.98 mmol, 1.0 eq), hydroxyamine hydrochloride (206 mg, 2.97 mmol, 1.5 eq) and potassium acetate (291 mg, 2.97 mmol, 1.5 eq) were dissolved in ethanol (5 ml)), it was stirred at 80° C. for 1 hour. After the reaction solution was cooled to room temperature, water (50 mL) was added and extracted three times with ethyl acetate (50 mL). An organic layer was washed with water, dehydrated with sodium sulfate (Na₂SO₄), filtered under reduced pressure, and concentrated. After the concentrate was dissolved in tetrahydrofuran (THF, 10 ml), LiAlH (150 mg, 3.96 mmol, 2.0 eq) was slowly added at 0° C. and stirred at 10° C. for 2 hours. After sodium sulfate hydrate (Na₂SO₄.10H₂O) was added to the reaction solution to complete the reaction, the reaction solution was filtered through a celite filter. The filtered solution was concentrated under reduced pressure to synthesize (5-phenethylfuran-2-yl)methanamine (52, 250 mg, crude) in the form of a yellow oil. ¹H NMR (CDCl₃, 400 MHz): δ (ppm) 7.26-7.30 (m, 2H), 7.17-7.22 (m, 5H), 6.00 (d, J=2.8 Hz, 1H), 5.88 (d, J=2.8 Hz, 1H), 3.78 (s, 2H), 2.90-2.97 (m, 4H).

Step 2) Synthesis of 2-oxo-2-(((5-phenethylfuran-2-yl)methyl)amino)ethyl acetate (53): After (5-phenethylfuran-2-yl) methanamine (52, 300 mg, 1.49 mmol, 1.0 eq) and diisopropylethylamine (DIEA, 384 mg, 2.98 mmol, 2 eq) were dissolved in dichloromethane (DCM, 5 ml), it was cooled to 0° C. After 2-chloro-2-oxoethyl acetate (245 mg, 1.79 mmol, 1.2 eq) was slowly added dropwise, the reaction solution was stirred at 10° C. for 2 hours. Water (50 mL) was added to the reaction solution and extracted twice with dichloromethane (DCM, 50 mL). An organic layer was washed with water, dehydrated with sodium sulfate (Na₂SO₄), filtered under reduced pressure, and concentrated. The concentrated solution was purified by column chromatography (PE/EA=5/1), and 2-oxo-2-(((5-phenethylfuran-2-yl)methyl)amino) ethyl acetate (53, 200 mg) in the form of a pure yellow oil was synthesized. ¹H NMR (CDCl₃, 400 MHz): δ (ppm) 7.26-7.30 (m, 2H), 7.17-7.22 (m, 3H), 6.40 (br s, 1H), 6.14 (d, J=3.2 Hz, 1H), 5.92 (d, J=3.2 Hz, 1H), 4.60 (d, J=4.4 Hz, 2H), 4.45 (d, J=5.6 Hz, 2H), 2.89-2.96 (m, 4H), 2.17 (s, 3H).

Step 3) Synthesis of 2-hydroxy-N-((5-phenethylfuran-2-yl)methyl)acetamide (ATB10099): After 2-oxo-2-(((5-phenethylfuran-2-yl)methyl)amino)ethyl acetate (53, 150 mg, 0.50 mmol, 1.0 eq) was dissolved in methanol (5 ml)/water (1 ml), LiOH.H₂O (21 mg, 0.10 mmol, 2.0 eq) was added and stirred at 25° C. for 16 hours. The reaction solution was concentrated under reduced pressure and purified by high-resolution liquid chromatography to synthesize 2-hydroxy-N-((5-phenethylfuran-2-yl)methyl)acetamide (ATB10099, 49 mg) in the form of pure white solid. ¹H NMR (CDCl₃, 400 MHz): δ (ppm) 7.27-7.31 (m, 2H), 7.17-7.22 (m, 3H), 6.60 (br s, 1H), 6.13 (d, J=2.8 Hz, 1H), 5.91 (d, J=2.8 Hz, 1H), 4.45 (d, J=5.2 Hz, 2H), 4.16 (s, 2H), 2.91-2.94 (m, 4H)., ESI-MS Calcd m/z for C₁₅H₁₇NO₃ [M]⁺ 259.3 Found 259.9.

### Preparation Example 17) the Compound of Example 20 was Synthesized by the Method Disclosed in Reaction Scheme 17 Below

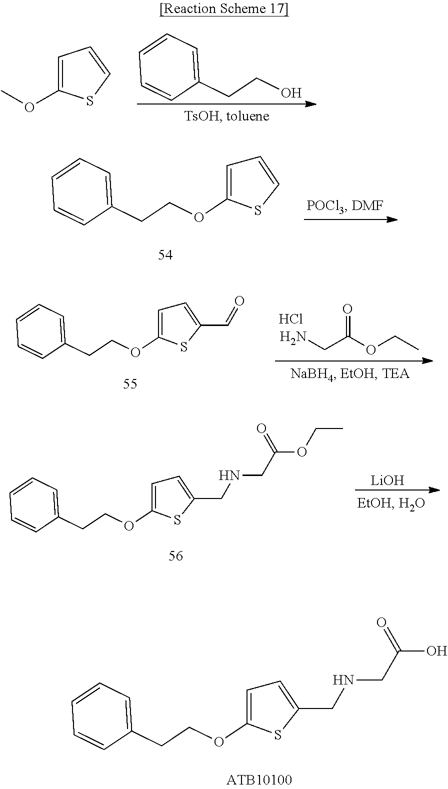

### Example 20: Preparation of ((5-phenethoxythiophen-2-yl)methyl)glycine (ATB10100)

Step 1) Synthesis of 2-phenethoxythiophene (54): After 2-methoxythiophene (1.0 g, 8.78 mmol, 1.0 eq) was dissolved in toluene (20 ml), 2-phenylethanol (2.7 g, 22.1 mmol, 2.5 eq) and p-toluenesulfonic acid (0.15 g, 0.87 mmol, 0.1 eq) were sequentially added at 10° C., and stirred at 90° C. for 1 hour. The reaction solution was put in water, extracted three times with ethyl acetate (EA, 30 ml), and then the organic layer was dehydrated with sodium sulfate (Na₂SO₄) and filtered under reduced pressure. The filtered solution was concentrated under reduced pressure and purified by column chromatography to synthesize 2-pheneoxythiophene (54, 0.9 g) in the form of a pure colorless oil. ¹H NMR (CDCl₃, 400 MHz): δ (ppm) 7.23-7.34 (m, 5H), 6.69-6.71 (m, 1H), 6.53-6.55 (m, 1H), 6.20-6.21 (m, 1H), 4.23 (t, J=6.8 Hz, 2H), 3.10 (t, J=6.8 Hz, 2H).

Step 2) Synthesis of 5-phenethoxythiophene-2-carbaldehyde (55): Dimethylformamide (DMF, 20 ml) was added to the reaction vessel, and POCl3 (1.69 g, 11.0 mmol, 5.0 eq) was slowly added while maintaining the temperature at 10° C., and then, stirred at the same temperature for 1 hour. 2-phenethoxythiophene (54, 0.45 g, 2.2 mmol, 1.0 eq) was dissolved in dimethylformamide (DMF, 2 ml) and slowly added dropwise to the reaction vessel at 10° C., and then stirred for 30 minutes. While the temperature in the reaction vessel was maintained at 0 to 5° C., 10N NaOH aqueous solution was added dropwise to the reaction solution until pH=9, and then stirred for 1 hour. Water (60 ml) was added to the reaction solution, extracted three times with ethyl acetate (EA, 30 ml), and the obtained organic layer was dehydrated with sodium sulfate (Na₂SO₄) and filtered under reduced pressure. The filtered solution was concentrated under reduced pressure and purified by column chromatography (PE/EA=3/1) to synthesize 5-phenethoxythiophene-2-carbaldehyde (55, 0.50 g) in the form of a pure yellow solid. ¹H NMR (CDCl₃, 400 MHz): δ (ppm) 9.66 (s, 1H), 7.50 (d, J=4.4 Hz, 1H), 7.25-7.36 (m, 5H), 6.33 (d, J=4.4 Hz, 1H), 4.34 (t, J=7.2 Hz, 2H), 3.14 (t, J=7.2 Hz, 2H).

Step 3) Synthesis of ethyl ((5-phenethoxythiophen-2-yl)methyl)glycinate (56): After 5-phenethoxythiophene-2-carbaldehyde (55, 300 mg, 1.3 mmol, 1.0 eq), glycine ethyl ester hydrochloride (360 mg, 2.59 mmol, 2.0 eq), triethylamine (262 mg, 2.59 mmol, 2.0 eq) and acetic acid (3 drops) were dissolved in ethanol (5 ml), it was stirred at room temperature for 16 hours. NaBH₄ (245 mg, 6.45 mmol, 5.0 eq) was added to the reaction solution, and then stirred at 60° C. for 16 hours. Then, water (20 ml) was added and extracted three times with ethyl acetate (EA, 30 ml). The extracted organic layer was washed with brine, dehydrated with sodium sulfate (Na₂SO₄), and filtered under reduced pressure. The filtered solution was concentrated under reduced pressure and purified by column chromatography (PE/EA=5/1) to synthesize ethyl ((5-phenethoxythiophen-2-yl)methyl)glycinate (56, 0.3 g, 72.7% yield) in the form of a pure yellow oil. ¹H NMR (CDCl₃, 400 MHz): δ (ppm) 7.24-7.36 (m, 5H), 6.54 (d, J=3.6 Hz, 1H), 6.04 (d, J=4.0 Hz, 1H), 4.18-4.25 (m, 4H), 3.88 (d, J=0.8 Hz, 2H), 3.43 (s, 2H), 3.11 (t, J=7.2 Hz, 2H), 1.30 (t, J=7.2 Hz, 3H).

Step 4) Synthesis of ((5-phenethoxythiophen-2-yl)methyl)glycine (ATB10100): After ethyl ((5-phenethoxythiophen-2-yl)methyl)glycinate (56, 300 mg, 0.94 mmol, 1.0 eq) was dissolved in ethanol (2 ml) and water (2 ml), LiOH (56 mg, 2.3 mmol, 2.5 eq) was added and stirred at room temperature for 2 hours. 2N HCl aqueous solution was added dropwise to the reaction solution until pH=7, stirred for 20 minutes, concentrated under reduced pressure, and purified by high-resolution liquid chromatography to synthesize ((5-phenethoxythiophene-2-yl)methyl)glycine (ATB10100, 50 mg) in the form of a pure white solid. ¹H NMR (DMSO_d₆, 400 MHz): δ (ppm) 7.22-7.34 (m, 5H), 6.67 (d, J=3.6 Hz, 1H), 6.18 (d, J=3.6 Hz, 1H), 4.23 (t, J=6.8 Hz, 2H), 3.90 (s, 2H), 3.11 (s, 2H), 3.03 (t, J=6.8 Hz, 2H)., ESI-MS Calcd m/z for C₁₅H₁₇NO₃S [M]⁺ 291.1 Found 290.1 [MS-1].

### Experimental Example 1. Evaluation of Oligomerization Activity of p62 Protein in Cultured Cells by Immunoblotting

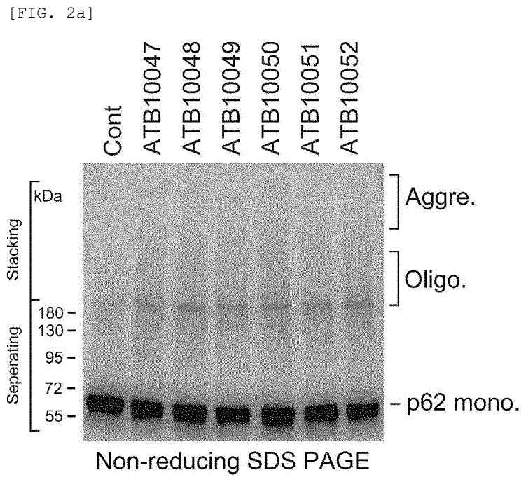

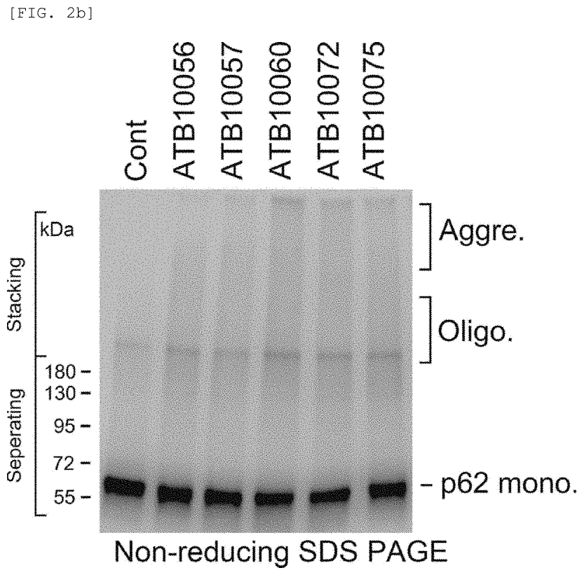

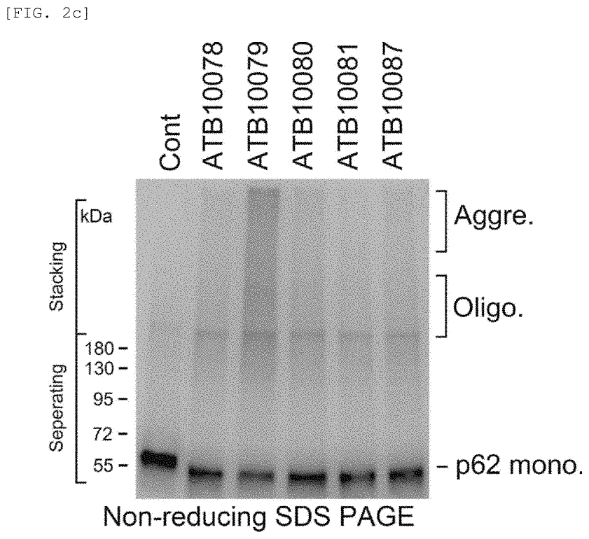

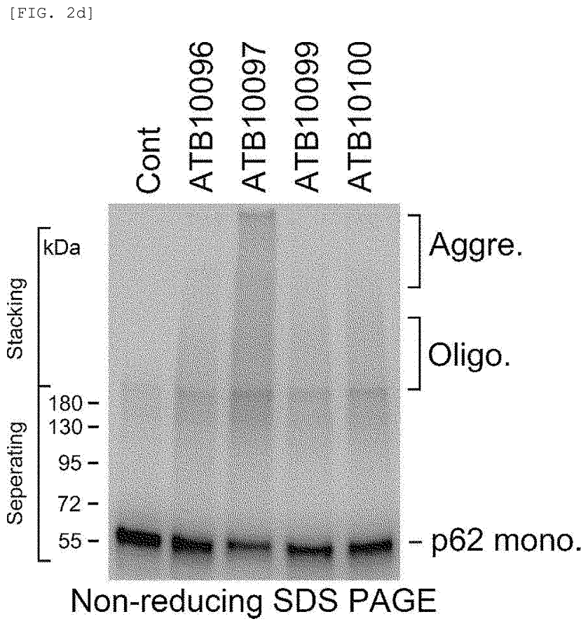

In order to evaluate the oligomerization activity efficacy of p62 protein of the compounds (Examples 1-20), HEK293 cell line, which is human embryonic kidney-derived cell, was collected. As representative compounds of the present compounds, the compounds of Examples 1-20 (Example 1 (ATB10048), Example 2 (ATB10047), Example 3 (ATB10049), Example 4 (ATB10051), Example 5 (ATB10050), Example 6 (ATB10056), Example 7 (ATB10052), Example 8 (ATB10057), Example 9 (ATB10060), Example 10 (ATB10072), Example 11 (ATB10075), Example 12 (ATB10078), Example 13 (ATB10079), Example 14 (ATB10080), Example 15 (ATB10081), Example 16 (ATB10087), Example 17 (ATB10096), Example 18 (ATB10097), Example 19 (ATB10099) and Example 20 (ATB10100)) were selected, and in order to measure the activation and oligomerization of the p62 protein in cells according to the treatment with these selected representative compounds, each cell was dispensed into a 100 phi dish. The cells were collected after additional culture for 24 hours so that the cells were completely attached to the surface of the plate, and 100 ul of lysis buffer (20 mM Tris (pH 7.4), 150 mM NaCl, 1% Triton X-100, 2 mM NaF, 2 mM EDTA, 2 mM beta-glycerophosphate, 5 mM sodium orthovanadate, 1 mM PMSF, leupeptin, aproteinin) was injected to each sample, and the cells were lysed. Based on the measured total protein concentration, each sample was treated with the test compounds for 2 hours at room temperature, and then a sample buffer was added and reacted at 95° C. for 10 minutes. 25 ul was taken from the samples after the reaction was completed and dispensed into each well of an acrylamide gel, and immunoblotting was performed. The immunoblotting showed representative results from three or more independent experiments. The results are shown in FIGS. 2a to 2d.

As can be seen in FIGS. 2a to 2d, when treated with the p62 ligand compounds according to the present invention, it was confirmed that the treatment with the compounds resulted in a decrease of the monomer of the p62 protein and simultaneously an increase in oligomers and high-molecular aggregates.

### Experimental Example 2. Evaluation of the Activity of p62 Protein and the Activity of Delivery of Ubiquitinated Substrate Proteins to Autophagy in Cultured Cells by Immunofluorescence Staining and Confocal Microscopy

Immunofluorescence staining was performed using p62 and FK2 as markers to determine the level of the activity of p62 protein of the compounds (Examples 1-20). In order to determine the level of p62 activity and autophagy activity by the novel p62 ligand and its isomers in the cultured cells, the Hela cell line derived from cervical cancer patients was treated with a novel p62 ligand compound (Example 1 (ATB10048), Example 2 (ATB10047), Example 3 (ATB10049), Example 5 (ATB10050), Example 4 (ATB10051), Example 7 (ATB10052), Example 6 (ATB10056), Example 8 (ATB10057), Example 9 (ATB10060), Example 10 (ATB10072), Example 11 (ATB10075), Example 12 (ATB10078), Example 19 (ATB10079), Example 14 (ATB10080), Example 15 (ATB10081), Example 16 (ATB10087)), Example 17 (ATB0096), Example 18 (ATB10097), Example 19 (ATB10099) and Example 20 (ATB10100)) and cultured, and then the level of puncta expression and location of FK2, which was a marker of the protein to be delivered to and degraded in the autophagy through the mediation of the intracellular ubiquitinated p62, and the puncta local co-existence with p62 were observed.

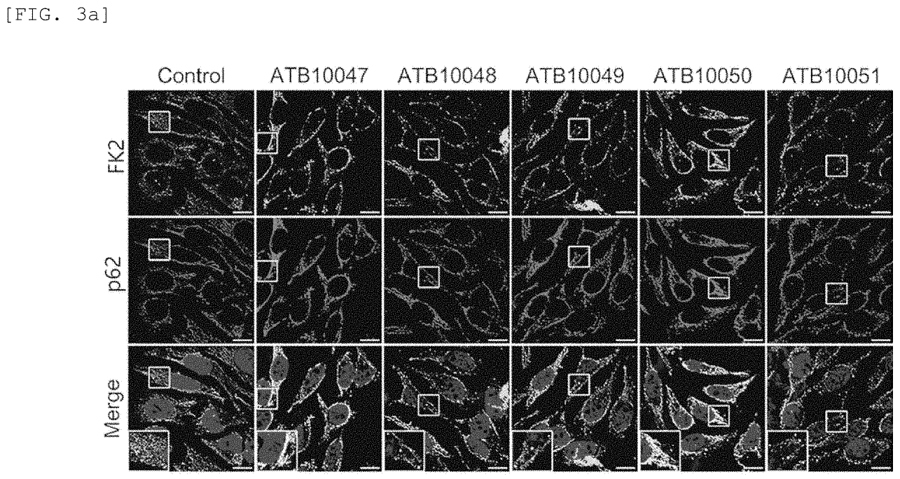

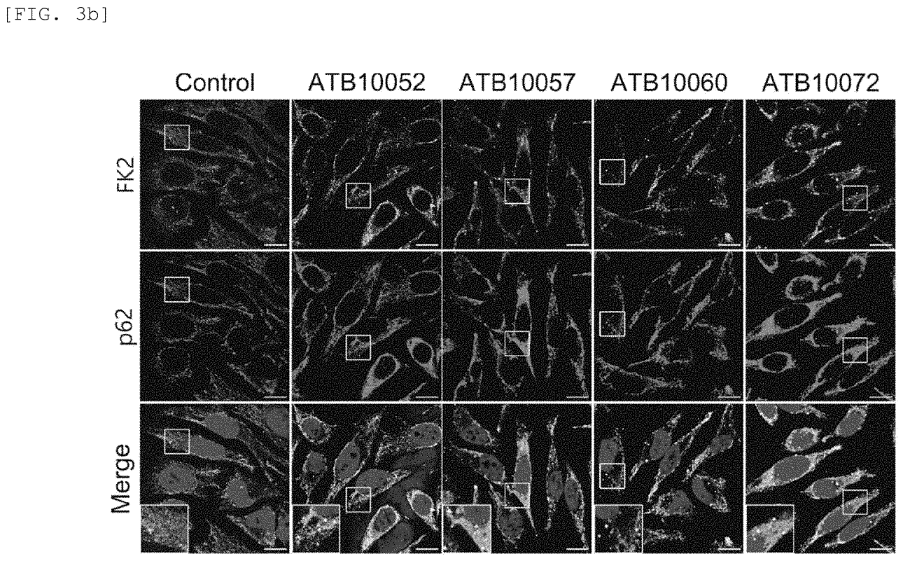

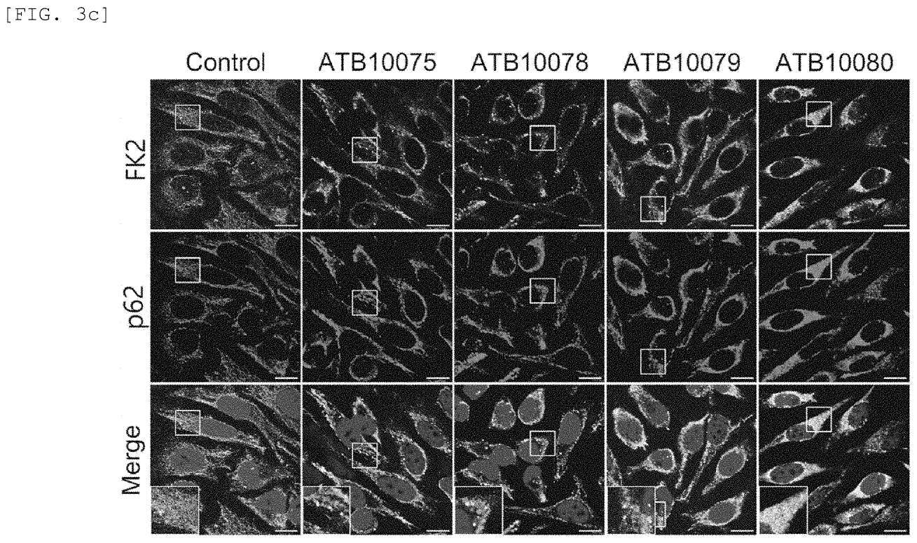

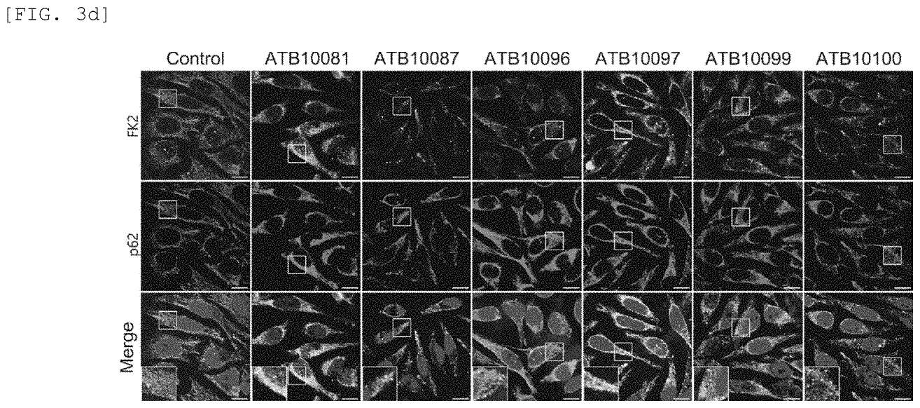

For immunofluorescence staining, a cover glass was placed on a 24-well plate, cells were dispensed and cultured for 24 hours, and then 1 uM of the novel p62 ligand according to the present invention was treated. After culturing for an additional 6 hours for the action of the compound, the medium was removed, and the cells were fixed using formaldehyde at room temperature. In order to prevent non-specific staining, the cells were reacted with a blocking solution at room temperature for 1 hour, and then the LC3 antibody diluted at a certain ratio was treated with a blocking solution and then reacted at room temperature for 1 hour. After the antibody-treated cells were washed 3 times with PBS, the goat-derived secondary antibody was diluted at a certain ratio using the blocking solution, and then reacted at room temperature for 30 minutes. After further washing with PBS three times, and DAPI staining was performed for intracellular nuclear staining. Then, the level of expression of p62 or FK2, the level of intracellular puncta formation, and the level of coexistence in the cells were observed by a confocal microscope. The results are shown in FIGS. 3a to 3d. Immunofluorescence staining showed representative results from three or more independent experiments.

As can be seen in FIGS. 3a to 3d, after treatment with the p62 ligand compounds according to the present invention, it was confirmed that the intracellular puncta formation of p62 proteins, intracellular puncta and local co-existence of FK2, which was a marker of substance proteins delivered to the autophagy through the mediation of the ubiquitinated p62 for degradation, and the intracellular puncta formation of FK2 were increased.

### Experimental Example 3. Evaluation of the Activity of p62 Protein and the Activity of Delivery to Autophagosome in Cultured Cells by Immunofluorescence Staining and Confocal Microscopy

Immunofluorescence staining was performed using p62 and FK2 marker to determine the level of activity of p62 protein of the compounds (Examples 1-20). In order to determine the level of p62 activity and autophagy activity by the novel p62 ligand and its isomers in cultured cells, Hela-LC3-GFP cell line derived from cervical cancer patients was treated with the novel p62 ligand compounds (Example 2 (ATB10047), Example 3 (ATB10049), Example 4 (ATB10051), Example 6 (ATB10056), Example 9 (ATB10060), Example 12 (ATB10078), Example 16 (ATB10087) and Example (ATB10097)) and cultured. Then, the level of puncta expression and location of LC3-GFP, which is an essential autophagosome marker for macroautophagy, and the puncta local co-existence of p62 were observed.

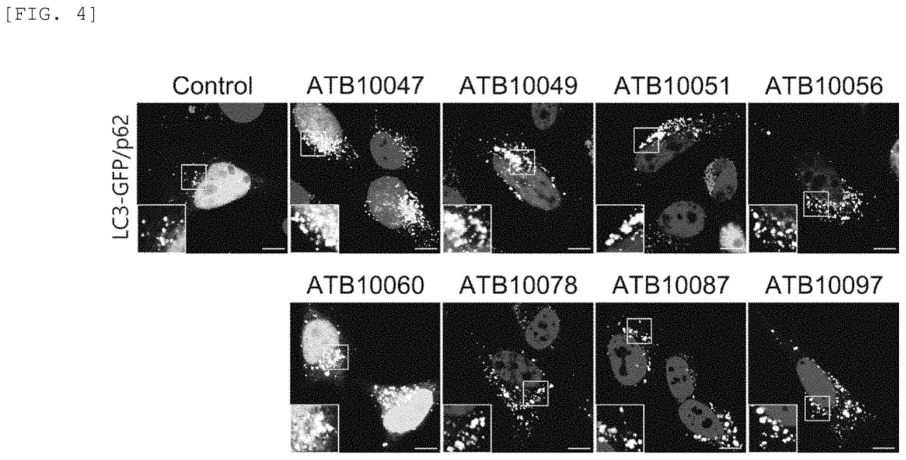

For immunofluorescence staining, a cover glass was placed on a 24-well plate, cells were dispensed and cultured for 24 hours, and then treated with 5 uM of the novel p62 ligand according to the present invention. The cells were further cultured for an additional 24 hours for the action of the compound, the medium was removed, and the cells were fixed using formaldehyde at room temperature. In order to prevent non-specific staining, the cells were reacted with a blocking solution at room temperature for 1 hour, and then the LC3 antibody diluted at a certain ratio was treated with the blocking solution and then reacted at room temperature for 1 hour. After the antibody-treated cells were washed 3 times with PBS, the goat-derived secondary antibody was diluted at a certain ratio using the blocking solution, and then reacted at room temperature for 30 minutes. The cells were washed again with PBS three times and subjected to DAPI staining for intracellular nuclear staining, and then the level of expression of p62 or LC3, intracellular puncta formation, and intracellular coexistence level were observed by a confocal microscope. The results are shown in FIG. 4. Immunofluorescence staining showed representative results from three or more independent experiments.

As can be seen in FIG. 4, it was confirmed that after the treatment with the p62 ligand compounds according to the present invention, intracellular puncta formation of the p62 protein, intracellular puncta and local co-existence of LC3, autophagosome marker, and intracellular puncta formation of LC3 were increased.

1. A p62 ligand compound of the following Chemical Formula 1, a pharmaceutically acceptable salt, stereoisomer, solvate, hydrate or prodrug thereof: 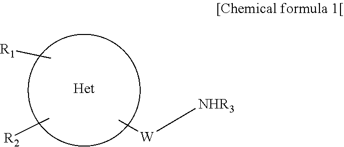
wherein, in Chemical Formula 1, the Het is a 4 to 10 membered heteroaryl or heterocyclyl comprising one or more heteroatoms selected from the group consisting of N, O and S;
R₁ and R₂ are each independently H, alkoxy having 1 to 4 carbon atoms, —NH—(CH₂)ₙ₁—R′, —O—(CH₂)ₙ₂—R′ or —(CH₂)ₙ₃—R′;
R′ is an aryl group having 6 to 10 carbon atoms;
W is a bond, —(CH₂)ₙ₄— or —O—(CH₂)ₙ₅—CH(OH)—(CH₂)ₙ₆—;
n1, n2, n3, n4, n5 and n6 are each independently an integer of 0 to 3; preferably an integer of 1 or 2; and
R₃ is a substituted or unsubstituted alkylene group having 1 to 4 carbon atoms, or acyl group having 1 to 4 carbon atoms, and a substituent of the substituted acyl group having 1 to 4 carbon atoms or alkylene group having 1 to 4 carbon atoms is —OH, —NH₂ or —COOR″, wherein, R″ is H or an alkyl group having 1 to 3 carbon atoms.

2. The compound, the pharmaceutically acceptable salt, stereoisomer, solvate, hydrate or prodrug thereof, according claim 1, wherein the Het is a 4 to 7 membered heteroaryl.

3. The compound, the pharmaceutically acceptable salt, stereoisomer, solvate, hydrate or prodrug thereof, according claim 1, wherein the R₁ and R₂ are each independently H, alkoxy having 1 to 3 carbon atoms, —NH—(CH₂)ₙ₁—R′, —O—(CH₂)ₙ₂—R′ or —(CH₂)ₙ₃—R′, wherein R′ is phenyl.

4. The compound, the pharmaceutically acceptable salt, stereoisomer, solvate, hydrate or prodrug thereof, according claim 1, wherein the n1, n2, n3, n4, n5 and n6 are each independently an integer of 1 to 3.

5. The compound, the pharmaceutically acceptable salt, stereoisomer, solvate, hydrate or prodrug thereof, according claim 1, 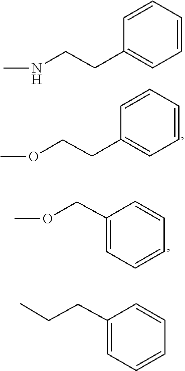
wherein the R₁ and R₂ are each independently —H, —OCH₃, —NHCH₂C₆H₅, —O(CH₂)₂C₆H₅, —OCH₂C₆H₅ or —(CH₂)₂C₆H₅.

6. The compound, the pharmaceutically acceptable salt, stereoisomer, solvate, hydrate or prodrug thereof, according claim 1, wherein the W is a bond, methylene, or —O—CH₂—CH(OH)—(CH₂)—.

7. The compound, the pharmaceutically acceptable salt, stereoisomer, solvate, hydrate or prodrug thereof, according to claim 1, wherein R₃ is a substituted or unsubstituted methylene, ethylene or propylene, or a substituted or unsubstituted acyl group having 1 to 3 carbon atoms,
wherein a substituent of the substituted acyl group having 1 to 3 carbon atoms or a substituent of the substituted methylene, ethylene or propylene is —OH, —NH₂, or —COOCH₃.

8. The compound, the pharmaceutically acceptable salt, stereoisomer, solvate, hydrate or prodrug thereof, according to claim 1, wherein the compound of Chemical Formula 1 is selected from the group consisting of the following compounds:
1) N-((6-benzyloxy)pyridin-2-yl)methyl)-2-hydroxyacetamide;
2)2-(((6-(benzyloxy)pyridin-2-yl)methyl)amino)ethan-1-ol;
3) N-((5-(benzyloxy)pyridin-2-yl)methyl)-2-hydroxyacetamide;
4) N-((4-(benzyloxy)pyridin-2-yl)methyl)-2-hydroxyacetamide;
5) 2-(((4-(benzyloxy)pyridin-2-yl)methyl)amino)ethan-1-ol;
6) 1-((5-(benzyloxy)pyridin-2-yl)methyl)urea;
7) N-((4,5-bis(benzyloxy)pyridin-2-yl)methyl)-2-hydroxyacetamide;
8) 2-(((4,5-bis(benzyloxy)pyridin-2-yl)methyl)amino)ethan-1-ol;
9) 2-(((5-(benzyloxy)pyrimidin-2-yl)methyl)amino)ethan-1-ol;
10) (R)-1-((4-(benzyloxy)pyridin-2-yl)oxy)-3-((2-hydroxyethyl)amino)propan-2-ol;
11) (R)-1-((6-(benzyloxy)-5-methoxypyridin-2-yl)oxy)-3-((2-hydroxyethyl)amino)propan-2-ol;
12) methyl (R)-3-((3-((4-(benzyloxy)pyridin-2-yl)oxy)-2-hydroxypropyl)amino)propanoate;
13) (R)-1-((5-(benzyloxy)pyridin-3-yl)oxy)-3-((2-hydroxyethyl)amino)propan-2-ol;
14) (R)-1-((6-(benzylamino)pyridin-2-yl)oxy)-3-((2-hydroxyethyl)amino)propan-2-ol;
15) (R)-1-((6-(benzyloxy)pyridin-2-yl)amino)-3-((2-hydroxyethyl)amino)propan-2-ol;
16) 2-(((5-phenethylfuran-2-yl)methyl)amino)ethan-1-ol;
17) 2-(((5-(benzyloxy)thiophen-2-yl)methyl)amino)ethan-1-ol;
18) 2-(((5-(benzyloxy)-furan-2-yl)methyl)amino)ethan-1-ol;
19) 2-hydroxy-N-((5-phenethylfuran-2-yl)methyl)acetamide; and
20) ((5-phenethoxythiophen-2-yl)methyl)glycine.

9. A composition comprising the p62 ligand compound, the pharmaceutically acceptable salt, stereoisomer, solvate, hydrate or prodrug thereof according to claim 1.

10-13. (canceled)

14. The composition according to claim 9, wherein the composition is a pharmaceutical composition or a food composition.

15-16. (canceled)

17. A method for increasing degradation of protein aggregates, comprising treating a cell or a p62 protein with the p62 ligand compound, the pharmaceutically acceptable salt, stereoisomer, solvate, hydrate or prodrug thereof according to claim 1.

18. A method for activating a selective autophagy, comprising treating a cell or a p62 protein with the p62 ligand compound, the pharmaceutically acceptable salt, stereoisomer, solvate, hydrate or prodrug thereof according to claim 1.

19. A method for preventing, ameliorating, or treating proteinopathy in a subject in need thereof, which comprises administering an effective amount of the p62 ligand compound, the pharmaceutically acceptable salt, stereoisomer, solvate, hydrate or prodrug thereof according to claim 1 to the subject.

20. The method according to claim 19, wherein the proteinopathy comprises neurodegenerative disease, anti-alpha 1 antitrypsin deficiency, keratopathy, retinitis pigmentosa, type 2 diabetes, or cystic fibrosis.

21. The method according to claim 20, wherein the neurodegenerative disease is one or more selected from the group consisting of Lyme borreliosis, Fatal familial insomnia, Creutzfeldt-Jakob Disease (CJD), multiple sclerosis (MS), dementia, Alzheimer's disease, epilepsy, Parkinson's disease, stroke, Huntington's disease, Picks disease, amyotrophic lateral sclerosis (ALS), spinocerebellar ataxia, other poly-Q diseases, hereditary cerebral amyloid angiopathy, familial amyloid polyneuropathy, primary systemic amyloidosis (AL amyloidosis), reactive systemic amyloidosis (AA amyloidosis), alpha1-antitrypsin deficiency, Alexander syndrome, keratopathy, retinitis pigmentosa, type 2 diabetes, cystic fibrosis, injection-localized amyloidosis, beta-2 microglobulin amyloidosis, hereditary non-neuropathic amyloidosis, Alexander disease and Finnish hereditary systemic amyloidosis.

22. The method according to claim 18, wherein the protein is one or more selected from the group consisting of a cancer-inducing protein, prion protein, amyloid precursor protein (APP), alpha-synuclein, superoxide dismutase, tau, immunoglobulin, amyloid-A, transtyretin, beta2-microglobulin, cystatin C, Apolipoproteine A1, TDP-43, islet amyloid polypeptide, ANF, gelsolin, insulin, lysozyme, fibrinogen, huntingtin, alpha-1-antitrypsin Z, crystallin, c9 open reading frame 72 (c9orf72), glial fibrillary acidic protein, cystic fibrosis transmembrane conductance regulator protein, rhodopsin and ataxin, and other proteins having a poly-Q stretch.

23. The method according to claim 19, wherein the p62 ligand compound, the pharmaceutically acceptable salt, stereoisomer, solvate or hydrate thereof is comprised as an active ingredient in a food composition.
